(19) United States
(12) Patent Application Publication
(10) Pub. No.: US 2007/0087383 A1
(43) Pub. Date: Apr. 19, 2007

(54) Immunoassay

(76) Inventors: Yinqiu Wu, Hamilton (NZ); John Stanton Mitchell, Rotorua (NZ)

Correspondence Address: JACOBSON HOLMAN PLLC, 400 SEVENTH STREET N.W., SUITE 600, WASHINGTON, DC, 20004, US

(21) Appl. No.: 10/572,678

(22) PCT Filed: Sep. 20, 2004
(86) PCT No.: PCT/NZ04/00222

§ 371 Date: Nov. 27, 2006

(30) Foreign Application Priority Data
Sep. 18, 2003 (NZ) 528323

(51) Int. Cl. G01N 33/53

(52) U.S. Cl. 435/7.1; 977/902

(57) ABSTRACT

The invention provides a method for detecting a hapten in a sample comprising the steps of: a) providing a sample potentially containing the hapten; b) providing a pre-determined amount of a first moiety, said first moiety being bound to a signaller and separated therefrom by a first linker, which first moiety is either: i) a binding partner that specifically binds to the hapten of interest, or ii) the hapten of interest or an analogue thereof; wherein said signaller is a macromolecule or a nanoparticle providing high mass signal; c) providing a flow of a) and b) separately or together to an immobilised second moiety, said second moiety being bound to the surface of a sensor and separated therefrom by a second linker, which second moiety is either: i) a binding partner that specifically binds to the hapten of interest, or ii) is the hapten of interest or an analogue thereof, providing that when the first moiety is a binding partner, the second moiety is a hapten or hapten analogue and when the first moiety is a hapten or hapten analogue, the second moiety is a binding partner; and d) detecting the amount of first moiety bound to second moiety.

### FIELD OF THE INVENTION

The present invention relates to a method for determination of haptens using a rapid flow-through immunoassay format.

### BACKGROUND

In sandwich or “catching antibody—antigen—labelled antibody” assays, two independent epitopes bound by different antibodies provide the advantages in terms of speed, sensitivity, and specificity. However, sandwich assay formats have not been directly applicable to small molecular weight haptens. Haptens are not large enough to bind simultaneously to two antibodies independently. For these reasons, competitive assays are the most widely used format for measurement of haptens.

To enhance assay sensitivities and specificities for haptens, non-competitive methods have been used. For example, anti-immune complex assays (Proc. Natl. Acad. Sci. USA, 90, 1993, 1184-1189 and Clin. Chem. 40(11), 1994, 2035-2041) were successfully used for determinations of tetrahydrocannabinol (THC) and digoxin. Selective antibody or ‘idiometric’ methodology (J. Immunol Methods 181, 1995, 83-90 and Steroids 60, 1995, 824-829) is another non-competitive approach, which provided more sensitive assays for estradiol and progesterone than conventional competitive enzyme assays. However, these non-competitive formats require unique antibodies and antiidiotypes that are potentially difficult to obtain. Another non-competitive two-site enzyme immunoassay format (hetero-two-site or immune complex transfer) (Biotechnology Annual Review 1, 1995, 403-451) has been also applied for small peptides or haptens with good detection levels. Unfortunately the immunoassay requires multiple steps. Multiple steps mean the assay is generally more expensive and time consuming than is desirable. The immunoassay also involves the use of harsh chemicals which potentially damage sensitive biomolecules and also involve the use of strongly acidic, basic or organic solvents that complicate providing assays in non-laboratory settings.

Another non-competitive assay for small molecules has been employed for measurement of cortisol and estradiol as described in U.S. Pat. No. 6,037,185. This assay permits the direct measurement of hapten bound sites or initial amount of hapten in the sample. Unfortunately, the assay still requires multiple steps to perform, which is potentially costly and time consuming.

Optical immunosensors are popular for bio-analysis. The non-destructive nature of the technology permits multiple reuses of samples for other readings. Rapid signal generation and thus rapid result generation are also advantages of the system. Unfortunately, label-free optical immunosensors have relatively poor analytical sensitivities to haptens with low molecular weight compared to traditional immunoassays such as ELISA. Despite significant developments in this field, optical immunosensors tend to be one magnitude less sensitive than commercial immuno assays for determining haptens.

It is an object of the present invention to provide an immunoassay that overcomes at least some of the above-mentioned disadvantages of existing assays; and/or that provides similar or better sensitivities to those of existing non-competitive formats; and/or that is rapid; and/or that has fewer steps than assays in the art, or that at least provides the public with a useful choice.

### DISCLOSURE OF THEE INVENTION

In a first aspect, the present invention provides a method for detecting a hapten in a sample comprising the steps of:
a) providing a sample potentially containing a hapten of interest;
b) providing a pre-determined amount of a first moiety, said first moiety being bound to a signaller and separated therefrom by a first linker, which first moiety is either:
   i. a binding partner that specifically binds to the hapten of interest, or
   ii. the hapten of interest or an analogue thereof;
   wherein said signaller is a macromolecule or a nanoparticle providing high mass signal.
c) providing a flow of a) and b) separately or together to an immobilised second moiety, said second moiety being bound to the surface of a sensor and separated therefrom by a second linker, which second moiety is either:
   i. a binding partner that specifically binds to the hapten of interest, or
   ii. is the hapten of interest or an analogue thereof,
   providing that when the first moiety is a binding partner, the second moiety is a hapten or hapten analogue and when the first moiety is a hapten or hapten analogue, the second moiety is a binding partner; and
d) detecting the amount of first moiety bound to second moiety.

In a further aspect, the present invention provides a method for detecting a hapten in a sample comprising the steps of:
a) providing a sample potentially containing a hapten of interest;
b) providing a predetermined amount of a binding partner that specifically binds to the hapten of interest, said binding partner being bound to a signaller and separated therefrom by a first linker wherein said signaller is a macromolecule or a nanoparticle providing a high mass signal;
c) providing a flow of a) and b) separately or together to an immobilised hapten of interest or an analogue thereof, said hapten or analogue thereof being bound to the surface of a sensor and separated therefrom by a second linker; and
d) detecting the amount of binding partner bound to said immobilised hapten or an analogue thereof.

In a still further aspect, the present invention provides a method for detecting a hapten in a sample comprising the steps of:
a) providing a sample potentially containing a hapten of interest;
b) providing a pre-determined amount of the hapten of interest or an analogue thereof, said hapten or analogue thereof being bound to a signaller and separated therefrom by a first linker wherein said signaller is a macromolecule or a nanoparticle providing a high mass signal;
c) providing a flow of a) and b) separately or together to an immobilised binding partner that specifically binds to the hapten of interest, said binding partner being bound to the surface of a sensor and separated therefrom by a second linker; and
d) detecting the amount of hapten or analogue thereof bound to said immobilised binding partner.

In a yet further aspect, the present invention provides a method for detecting a hapten in a sample comprising the steps of:
a) providing a sample potentially containing a hapten of interest;
b) providing a pre-determined amount of a first moiety, said first moiety being bound to a signaller, which first moiety is either:
   i. a binding partner that specifically binds to the hapten of interest, or
   ii. the hapten of interest or an analogue thereof;
      wherein said signaller is a macromolecule or a nanoparticle providing a high mass signal;
c) providing a flow of a) and b) separately or together to an immobilised second moiety, said second moiety being bound to sensor surface, which second moiety is either:
   i. a binding partner that specifically binds to the hapten of interest, or
   ii. is the hapten of interest or an analogue thereof,
   providing that when the first moiety is a binding partner, the second moiety is a hapten or hapten analogue and when the first moiety is a hapten or hapten analogue, the second moiety is a binding partner; and
d) detecting the amount of first moiety bound to second moiety, characterised in that said first moiety is bound to and separated from said signaller by a first linker and said second moiety is bound to and separated from said immobilisation substrate by a second linker.

In another aspect, the present invention provides a kit for determining the presence of a hapten of interest in a sample, which kit at least includes:
a) a first moiety being bound to a macromolecule or a nanoparticle and separated therefrom by a first linker, which first moiety is either:
   i. a binding partner that specifically binds to the hapten of interest, or
   ii. the hapten of interest or an analogue thereof; and
b) a sensor with an immobilised second moiety, said second moiety being bound to the sensor and separated therefrom by a second linker, which second moiety is either:
   i. a binding partner that specifically binds to the hapten of interest, or
   ii. is the hapten of interest or an analogue thereof,
   providing that when the first moiety is a binding partner, the second moiety is a hapten or hapten analogue and when the first moiety is a hapten or hapten analogue, the second moiety is a binding partner.

In another aspect, the present invention provides a kit for determining the presence of a hapten of interest in a sample, which kit at least includes:
a) a first moiety being bound to a signaller, which first moiety is either:
   i. a binding partner that specifically binds to the hapten of interest, or
   ii. the hapten of interest or an analogue thereof;
      wherein the signaller is a macromolecule or a nanoparticle; and
b) a sensor with an immobilised second moiety, said second moiety being bound to the sensor, which second moiety is either:
   i. a binding partner that specifically binds to the hapten of interest, or
   ii. is the hapten of interest or an analogue thereof,
   providing that when the first moiety is a binding partner, the second moiety is a hapten or hapten analogue and when the first moiety is a hapten or hapten analogue, the second moiety is a binding partner,
      characterised in that said first moiety is bound to and separated from said signaller by a first linker and said second moiety is bound to and separated from said immobilization substrate by a second linker.

In preferred embodiments of the above aspects of the invention the sample a) and the predetermined amount of the second moiety b) are mixed and in step c) the mixture is caused to flow to the immobilised second moiety.

In one embodiment, the present invention provides a method for detecting a hapten in a sample using a rapid flow-through inhibition assay format comprising the steps of:
a) Providing a functionalised hapten derivative with a linking group (first linker) between the hapten molecule and its functional group;
b) Providing an immobilised hapten derivative on the surface of an optical biosensor chip wherein the hapten derivative is linked to the surface through a linking group (first linker) between the hapten molecule and the surface;
c) Mixing high molecular weight detecting molecules, for example antibodies, with sample analytes to form immuno-complexes, and then providing flow-through of the mixing solution containing excess free antibodies to bind to the sensor surface;
d) Further binding enhancement performed by flowing-through onto the sensor surface with a solution containing a conjugate employing a linker (second linker), a moiety to specifically recognise a detecting molecule such as an antibody is linked at one end of the conjugate, and the other end of the conjugate is attached to a protein or/and a nano-particle for high mass signal enhancement;

In another embodiment, the present invention provides a rapid flow-through competition immunoassay method for detecting a hapten in a sample comprising the steps of:
a) Providing immobilised detecting molecules for example antibodies on the biosensor surface with a linker (first linker) between a biomaterial as an attachment intermediate and the detecting molecule;
b) Mixing sample analytes with a hapten conjugate, in which a protein or/and a nano-particle is linked to the hapten molecule with a linker (second linker) and having a nano-distance (nm) between the protein/nano-particle and the hapten molecule to reduce steric hindrance;
c) Flowing through the mixture of hapten conjugate and sample analyte solution onto the sensor surface for binding competition to limited detecting molecules such as antibodies on the surface of the sensor;

In preferred embodiments, rapid on-line regeneration is used to completely remove hapten conjugates to allow multiple measurements. This may be carried out by injection of regeneration solutions that may include sodium hydroxide and acetonitrile.

A standard curve may be prepared from solutions with a series of known analyte concentrations, and the concentrations of analyte in unknown samples may then derived from the standard curve.

The present invention includes a new design based on a: novel concept of Dual-Linker Technology with High Mass Labelling (FIG. 1) for flow-through optical biosensors such as Surface Plasmon Resonance (SPR) based immunoassays to achieve high binding signal and assay sensitivity enhancement particularly for small molecular weight analytes, such as therapeutic and abused drugs, steroids, thyroid hormones, metabolites and pollutants etc.

As stated above, the present invention provides, in a first aspect, a method for detecting a hapten in a sample. The method comprises several essential steps.

The first step is providing a sample potentially containing a hapten of interest. A pre-determined amount of a first moiety is provided. The first moiety is provided bound to the signaller and separated therefrom by a first linker. The first moiety is either a binding partner that specifically binds to the hapten of interest or the hapten of interest or an analogue thereof.

The two components or a mixture thereof is now contacted with an immobilised second moiety. The second moiety is provided bound to the detection surface of a sensor and separated therefrom by a second linker. The second moiety is either a binding partner that specifically binds to the hapten of interest, or is the hapten of interest or an analogue thereof. However, when the first moiety is a binding partner, the second moiety must be a hapten or hapten analogue. Alternatively, when the first moiety is a hapten or hapten analogue, the second moiety must be a binding partner. The amount of first moiety bound to second moiety is then detected.

The linker can be bound directly to the detection surface of a sensor, for example by a covalent bond formed from an amine group at the end of the linker and a carboxyl group on the surface. Alternatively the linker may be bound to another molecule for example a protein (for example ovalbumin) which may bind to the surface. Thus the linker may connect directly with the surface or other components may be inserted between the first moiety and the surface.

In the context of this invention, the term “hapten” means any small molecular hapten which has a molecular weight less than 5000 Daltons. Most usually, the hapten is an organic compound of low molecular weight (less than 2000 Daltons) that reacts specifically with an antibody and which is incapable of eliciting an immune response by itself but is immunogenic when complexed with an antigenic carrier. Haptens of interest here are selected from the group comprising carbohydrates, polynucleotides, steroids, steroid analogues, polypeptides (such as peptide hormones), drugs and toxins, but are not limited thereto. Haptens of particular interest in the present invention include therapeutic drugs, narcotics, steroids, thyroid hormones, metabolites and pollutants. The invention has particular application with smaller haptens as steric hindrance caused by attachment is more of a problem with smaller haptens.

Herein, “binding partner” refers to macromolecules capable of specifically binding to a target hapten of interest. Examples of suitable macromolecules include antibodies and fragments thereof as well as nucleic acids, such as an RNA aptamer described in Biochemical and Biophysical Research Communications 281, 237-243 (2001) and incorporated herein by reference.

Antibodies are well known to those of ordinary skill in the science of immunology. As stated above, included within the ambit of “binding partner” are not only intact antibody molecules but also fragments of antibody molecules retaining hapten-binding ability. Such fragments are also well known in the art and are regularly employed both in vitro and in vivo.

Therefore, “binding partner” also includes not only intact immunoglobulin molecules but also the well-known active fragments F(ab′)₂, and Fab. F(ab′)₂, Fab fragments which lack the Fc fragment of intact antibody, Fv, single chain (ScFv), mutants thereof, fusion proteins comprising an antibody portion, and any other modified configuration of the immunoglobulin molecule that comprises an antigen recognition site of the required specificity. In an alternative embodiment, the binding partner may be a T-cell receptor. Other types of binding protein may be used where these can be identified, and have sufficient specificity for the hapten of interest.

“specifically binds” or “specifically binding” in the present invention means that the binding partner binds to the hapten of interest without substantial cross reactivity to other species in the sample to enable a meaningful detection result to be obtained.

“analogue” of a hapten herein refers to a group that competes with the hapten for binding to a binding partner. In the case of antibodies, the analogue should bind to the same site on the antibody as the hapten.

“sample” is typically a liquid sample from a biological source, but is not limited thereto.

“surface of a sensor” is the surface of any bulky suitable substantially insoluble support forming part of a sensor that permits attachment of a linker. The surface may include but is not limited to a chip surface, gels (e.g. cross-linked chromatography gels) and a solid support as well as any other support well known in the art. Non-limiting examples of suitable immobilisation substrates suitable for the practice of the present invention include:

(a) insoluble polymeric materials such as polystyrene, polypropylene, polyester, polyacrylonitrile, polyvinyl chloride, polyvinylidene, polysulfone, polyacrylamide, cellulose, cellulose nitrate, cross-linked dextrans, fluorinated resins, agarose, crosslinked agarose, and polysaccharides etc;

(b) glass, glass fibres, and glass beads;

(c) metal (gold, silver or platinum), metal strips and metal beads;

(d) nylon mesh material and nylon membranes; and

(e) test tubes, microtiter plates, dipsticks, lateral flow devices, resins, PVC, latex beads and nitrocellulose.

Preferably the sensor is based around a surface of an optical biosensor chip. Preferably the chip is adapted for use in an optical system in which high mass groups can be detected on a surface. Most preferably the chip is adapted for use in a surface plasmon resonance detection system.

A preferred sensor chip is a BIAcore CM5 chip.

The invention is directed to “rapid” assays, characterised in that they are flow-through or flow-over assay formats, giving rapid signal generation and a reading typically in less than 10 minutes. The invention is particularly suited to a rapid flow-through assay using a commercial BIAcore instrument.

In one embodiment of the present invention, hapten molecules are chemically immobilised onto a sensor surface with a linker interposed between the hapten and the surface. In an alternative embodiment, the hapten is attached to an attachment intermediate material with a linker interposed between the hapten and the attachment intermediate material. The attachment intermediate is, in turn, attached to a sensor surface. Preferred attachment intermediates are selected from the group comprising proteins (Steroids, 67, 2002, 565-572), nucleic acid fragments (U.S. Pat. No. 5,849,480) and N-vinylpyrrolidone copolymer (U.S. Pat. No. 5,723,334). Examples of suitable proteins as attachment intermediate materials include bovine serum albumin (BSA), ovalbumin (OVA) or keyhole limpet hemocyanin (KLH). Proteins may also include enzymes, secretory proteins, globular proteins. A preferred protein for use herein is ovalbumin (OVA).

Where the hapten is a steroid, it is preferred that binding of the hapten to the linker occurs at the 4-position of the structure. The binding at the 4-position of the A ring is particularly preferred when binding estrogens, progesterone and steroids having an A-ring structure similar to progesterone. Moieties of formulae 14-17, 20-23 and 29-32 are currently preferred steroids for binding at the 4-position on the A ring (see Examples).

When the hapten is an aromatic neurotransmitter molecule such as dopamine or serotonin, it is preferred that binding of the hapten occurs at the aromatic ring.

In the currently most preferred embodiment, the hapten is progesterone.

The “first linker” and “second linker” are typically each independently 4 to 50 atoms in length, preferably 10 to 50, more preferably 10 to 30 atoms in length excluding any bridging groups. Linkers suitable for the practice of the present invention are preferably (a) a carbon-based chain; (b) carbon-chain containing one or more heteroatoms such as N, S, O; (c) carbon-chain with substituted groups; (d) an amino acid chain, amino acid fragments incorporated into the chain, or multiple amino-acid fragments chain by for example homologation; (e) a polyethylene glycol chain; (f) a chain have one or more sites of unsaturation such as alkenyl; (g) a nucleic acid chain; or (h) a polysaccharide chain etc. Obviously, depending on the nature and physical size of the moiety attached to the chain, the chain can be made hydrophobic or hydrophilic by including fewer or more groups respectively that are more polar or ionic in the chain.

The second linker can be selected from different molecular types and lengths. It has been found that the best performance is obtained when the second linker is selected to ensure that non-bulky groups are proximal the hapten. It is preferred that the chain be carbon-based. The carbon-based chain may comprise one or more heteroatoms selected from N, S, and O. Side chain substituent groups may also be provided. Other preferred chains are selected from the group comprising amino acids, a polyethylene glycol, alkyl, alkenyl, nucleic acid, and polysaccharide. The chain can have one or more sites of unsaturation. Multiple amino-acid fragments may be provided by homologation. The use of hybrid peptide-nucleic acid fragments as linkers is also contemplated.

The use of nano-sized “dual linker” or a first linker—between the chip surface and the centre of the immuno-complex, and a second linker—between the centre of immuno-complex and a large protein or/and a nano-particle will greatly reduce the steric hindrance to enhance antibody binding, and hence increases the assay sensitivities, assay speed and easy regeneration for multiple measurements. Typically each linker provides a chain of length 0.5-100 nm, preferably most preferably 1-5 nm.

One preferred synthesis of the first and second linkers for use in the present invention in different length is controlled and performed by successive aminocaproic acid homologation of hapten acid derivatives as illustrated in Reaction Scheme 1 before conjugation to proteins or immobilised onto the sensor surface directly.

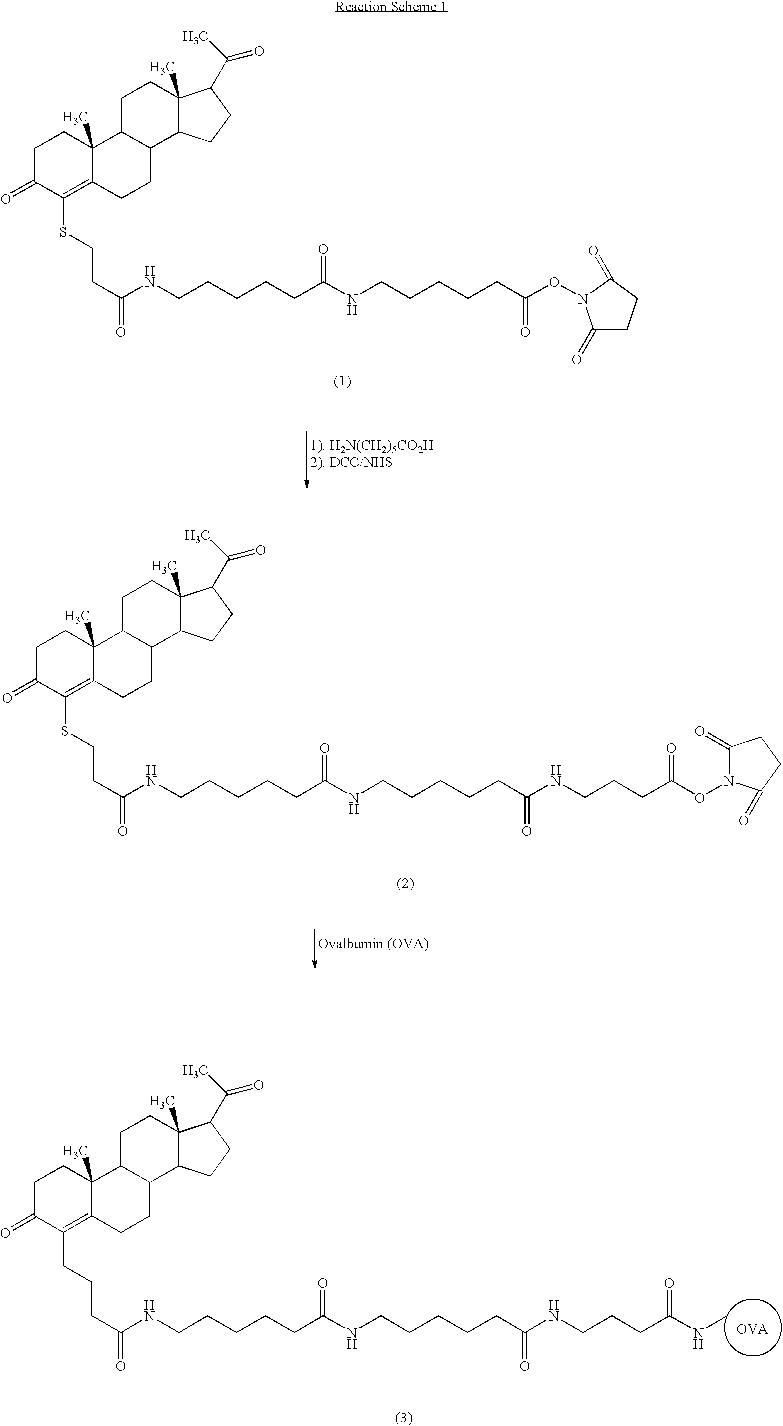

The structure of progesterone-ovalbumin conjugate with a 25-atoms linker (3), and its synthesis from the conjugate (4) (Steroids, 67, 2002, 565-572). The conjugate (3) was immobilised onto the SPR biosensor surface.

A more preferred synthesis of a hapten derivative to use in the present invention is controlled and performed by inserting a polyethylene glycol (PEG) chain in different length as a linker and immobilised the hapten derivative onto the sensor surface directly (Reaction Scheme 2). Such hapten derivative having a PEG unit as a linker has some distinctive advantages such as 1) PEG chain as a linker can make hapten derivative more water-soluble, and therefore the hapten derivative can be easily in-situ or on-line immobilized onto the sensor surface, which is convenient in real time for process monitoring and quality control in terms of reproducibility performance of immobilization. Use of a PEG chain as a linker can also provide hydrophilic molecular layers to reduce non-specific binding and create more space and a favourable binding medium between the chip surface and the immuno-complex for better antibody binding.

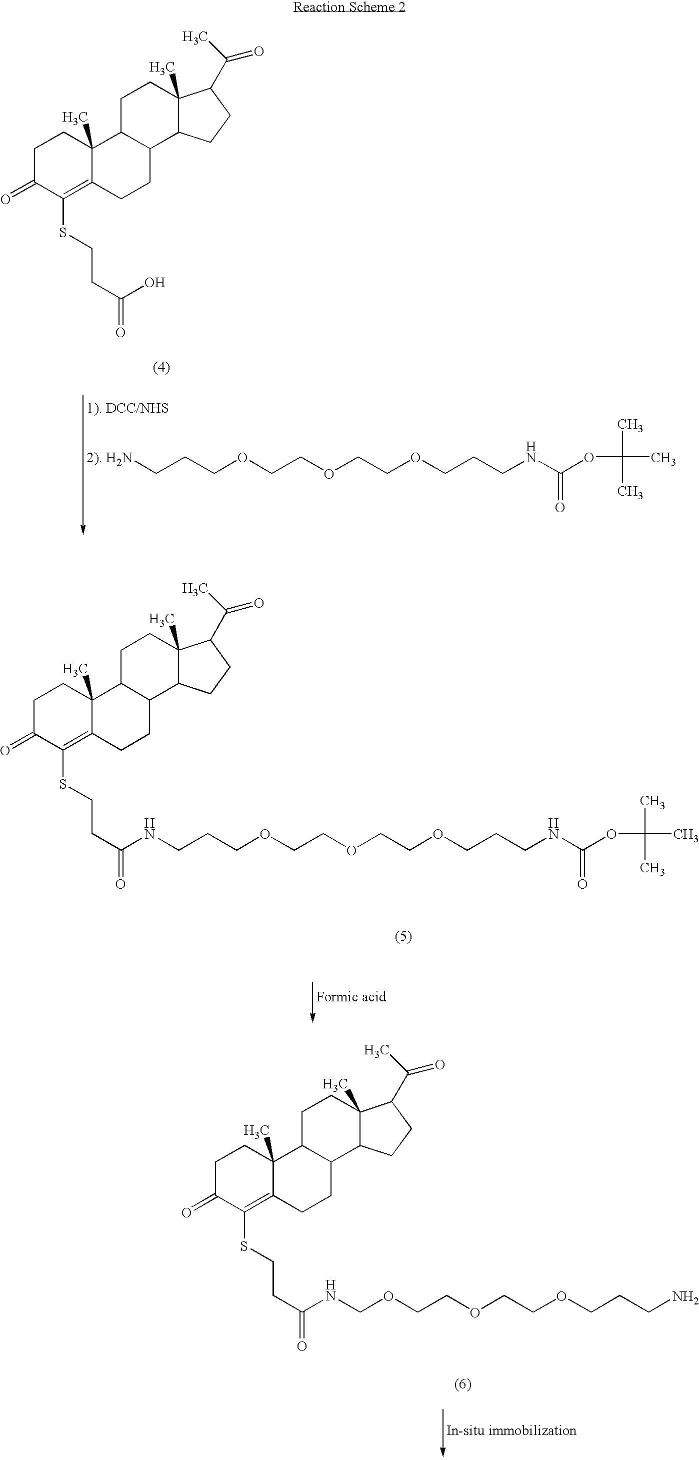

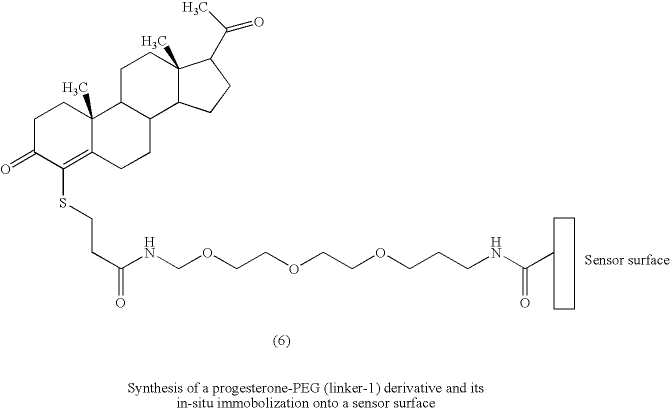

The progesterone-PEG (linker-1) derivative of Reaction Scheme 2 may be synthesised from progesterone-4-thiopropanoic acid (1) (Steroids, 67, 2002, 565-572) and in-situ immobilized onto a sensor surface.

There are many well-known immobilisation techniques in the art. Preferred immobilisation techniques for immobilising the first moiety, hapten to be immobilised or binding partner to be immobilised onto a sensor surface is by a covalent coupling reaction (e.g. to an amine, a carboxyl or sulfhydryl group on the protein), nucleic acid hybridisation, or ligand interaction. Immobilisation on the sensor surface may be also by passive adsorption, or via a ligand interaction, such as an avidin/biotin complex (U.S. Pat. No. 4,467,031).

Any suitable linker known in the art may be employed. Other examples of hapten-linker molecules useful in the practice of the present invention having different end-functional groups are shown Formulae 14-17, 20-23, 29-32, 34, 35, 37 and 38 (see Examples).

In order to covalently bind hapten to first and second linking groups in the practice of the present invention, it is often necessary to include a thioether or ether bridging group, preferably a thioether group, generally through their mono-bromide intermediate compounds.

“signaller” herein means a group capable of providing high mass labels for signal enhancement. Preferred embodiments include large proteins of molecular weight at least 20 kD, preferably at least 50 kD, more preferably at least 100 kD and nanoparticles (metal or non-metal; colour or non-colour) such as immunogold and coloured latex beads. Preferably the nanoparticles have a diameter/long axis of 1 nm-1000 nm, preferably 10-500 nm most preferably 10-20 nm.

The term “nanoparticles” refers to the particles used to provide sensitivity through mass labels and are solid particles ranging widely in the size of nanoscale, which includes metal particles (colloidal gold), non-metal particles (latex beads), or any other suitable nanoparticles used as mass labels for signal enhancement.

The term “macromolecule” refers to a molecule with a molecular weight of at least 20 kD. Macromolecules for use as signallers in this invention are preferably of molecular weight 50 kD, more preferably at least 100 kD.

Detecting the amount of bound double linker moieties of the present invention may be undertaken utilising a number of different techniques available in the art.

In one embodiment, immunogold particles are used because they are inexpensive and relatively stable.

The inventors have discovered that provision of a double linker molecule of the present invention increases binding partner binding performance in short-duration assays, such as flow-through assays leading to better assay sensitivities than with single linker or no linker systems. It has also been discovered that a most preferred detection system, surface plasmon resonance (SPR) utilising nano-particles gives unexpectedly good sensitivities when used in conjunction with double linker technologies.

It has also been found by the inventors that the use of double linkers in the methods of the present invention permits easier regeneration of a detection system for multiple readings.

In a currently preferred embodiment, a streptavidin/biotin linkage with a short aminocaproic acid chain conjugate 9 (see Reaction Scheme 3) is used in the construction of the first linker between a binding partner and a nanoparticle, which is 10 nanometres in size. When a large size of nanoparticle such as a 20 nm bead is used, the first linker should preferably be designed much longer for consideration of easy regeneration on the sensor surface.

In a preferred embodiment, the present invention relates to a new design of optical biosensor-based competitive immunoassays (FIG. 1) particularly surface plasmon resonance (SPR)-based immunoassays for small molecular weight haptens, such as therapeutic and abused drugs, steroids, thyroid hormones, metabolites and pollutants. This SPR-based immunoassay format method comprises the steps:
(a). chemically immobilising hapten (A) or hapten conjugate onto the optical biosensor surface through a linker molecule (the second linker) with or without using a hapten attachment intermediate,
(b). mixing a fixed concentration of binding partner (B)—(the first linker)—nanoparticle conjugate in buffer with each of a series of standard free solution or a sample hapten solution and incubating for a few minutes,
(c). injecting the above mixture or the remaining binding partner (B) in equilibrium solution onto the hapten (A) biosensor surfaces, and measuring binding partner (B) responses,
(d). injecting regeneration buffer, preferentially composed of sodium hydroxide and acetonitrile onto the biosensor surface to remove binding partner-(the first linker)-nanoparticle conjugate,
(e). plotting concentrations of free hapten versus average response (RU) of binding partner—(the first linker)—nanoparticle conjugate to provide an assay standard curve from which determining the concentration of unknown sample hapten when using the same method.

It is preferred that steps (b), (c) and (d) are repeated three times or more for reproducibility.

With reference to FIG. 1, the currently most preferred embodiment of the invention is now described. Design of “dual-linker” and “nanoparticle” is: (1) For hapten conjugate; Amino group—linker (10˜30 atoms in length) (thiopropanoic acid with 1˜3 minocaproic acids)—small molecular hapten (progesterone); (2) For binding partner conjugate; antibody—long linker (anti-IgG)—gold nanoparticle (10 nanometre) (Reaction Scheme 3). Based on the above design, a rapid flow-through (BIAcore 2000) and sensitive immunoassay for small molecular hapten (progesterone, MW=314.47) is achieved. The lowest detection limit (LDL) for the assay is around 8.6 pg/ml or 0.027 pM (2.7×10⁻¹⁴° M.).

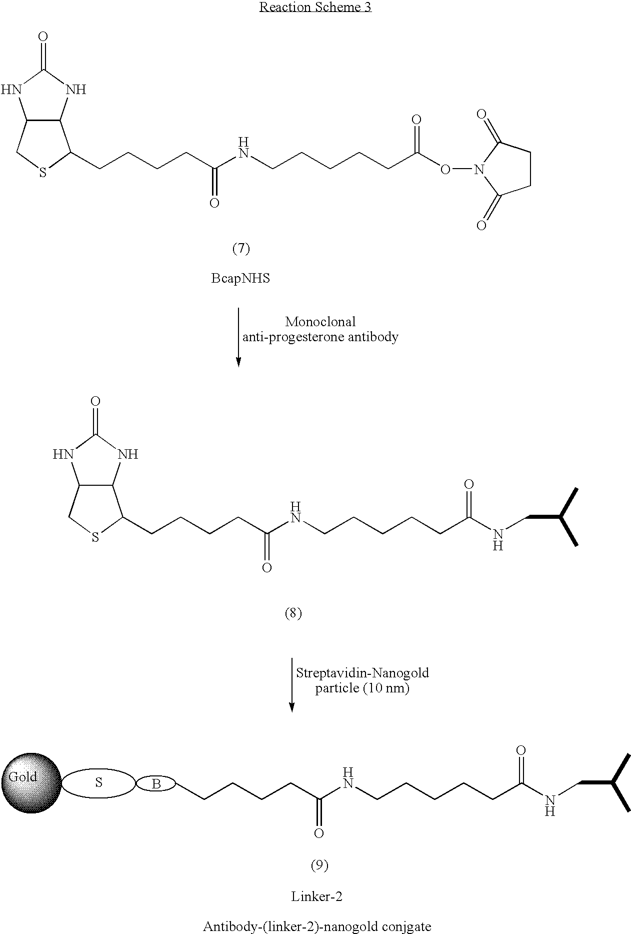

This reaction scheme shows the structure of antibody-(linker-2)-nanogold conjugate (9) through the biotin/streptavidin linkage, and its preparation from commercial biotin agent BcapNHS (7) with monoclonal anti-progesterone antibody (B) and followed by reaction with commercial streptavidin-nanogold particles (10 nm).

Based on the concept of a dual-linker combined with nanoparticle enhancement, the use of all other variations on the above methods by, for example, including various nanoparticles in different sizes, different types, lengths, and molecular hybridisations of dual linkers fall within the scope of the present invention.

The invention also extends to kits comprising a first and a second moiety with their various attachments as described above in separate containers with or without instructions for their use.

The invention is illustrated by the following non-limiting examples.

### BRIEF DESCRIPTION OF THE DRAWINGS

FIG. 1 shows a rapid optical biosensor-based immunoassay format using “dual-linker design with nanoparticle enhancement”.

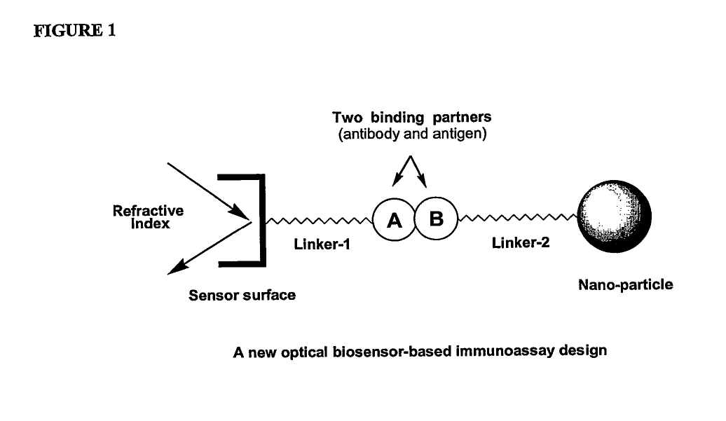

FIG. 2 shows the standard curve (RU percentage value to RU at 0 progesterone concentration versus concentration of progesterone in the range 0 to 1 μg/ml measured according to the method of this invention.

FIG. 3 shows a sensorgram for monoclonal anti-progesterone antibody binding (25 μg/mL) followed by anti-IgG (secondary antibody) binding enhancement (800 μg/mL) and regeneration.

FIG. 4 shows low binding responses of monoclonal anti-progesterone antibody (♦) and sequential anti-IgG (secondary antibody) enhanced binding (▪).

FIG. 5 shows a biotin/streptavidin mediated gold enhancement binding curve [response (RU) verse antibody/gold volume ratio] for a pre-incubation format.

FIG. 6 shows a standard curve for a pre-incubation method of biotin/streptavidin mediated nanogold enhanced immunoassay.

FIG. 7 shows comparisons of three standard curves using a sequential binding format of biotin/streptavidin mediated nanogold enhanced immunoassay with three different concentrations of biotinylated monoclonal antibody [(♦) 2.5 μg/mL, (▪) 7.5 μg/mL, and (▴) 15 μg/mL].

### EXAMPLES

Syntheses of Hapten Derivatives

The structures of relevant compounds include:

### Progesterone and Progesterone Analogue

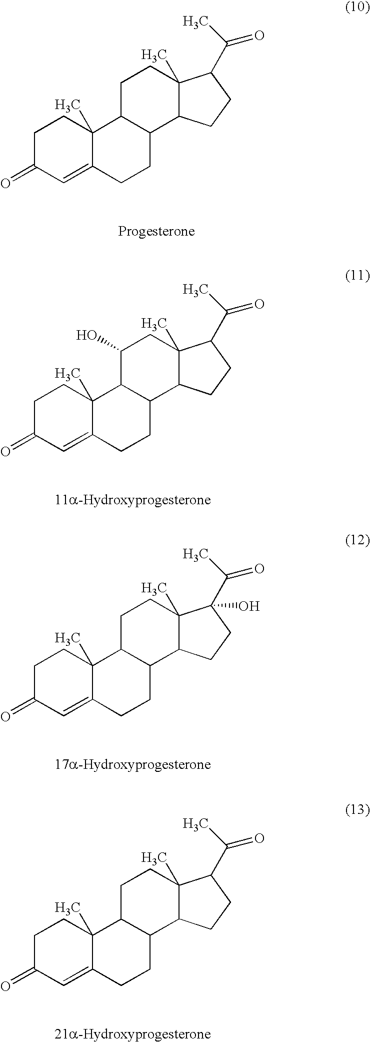

### Progesterone-4-Positional Derivatives

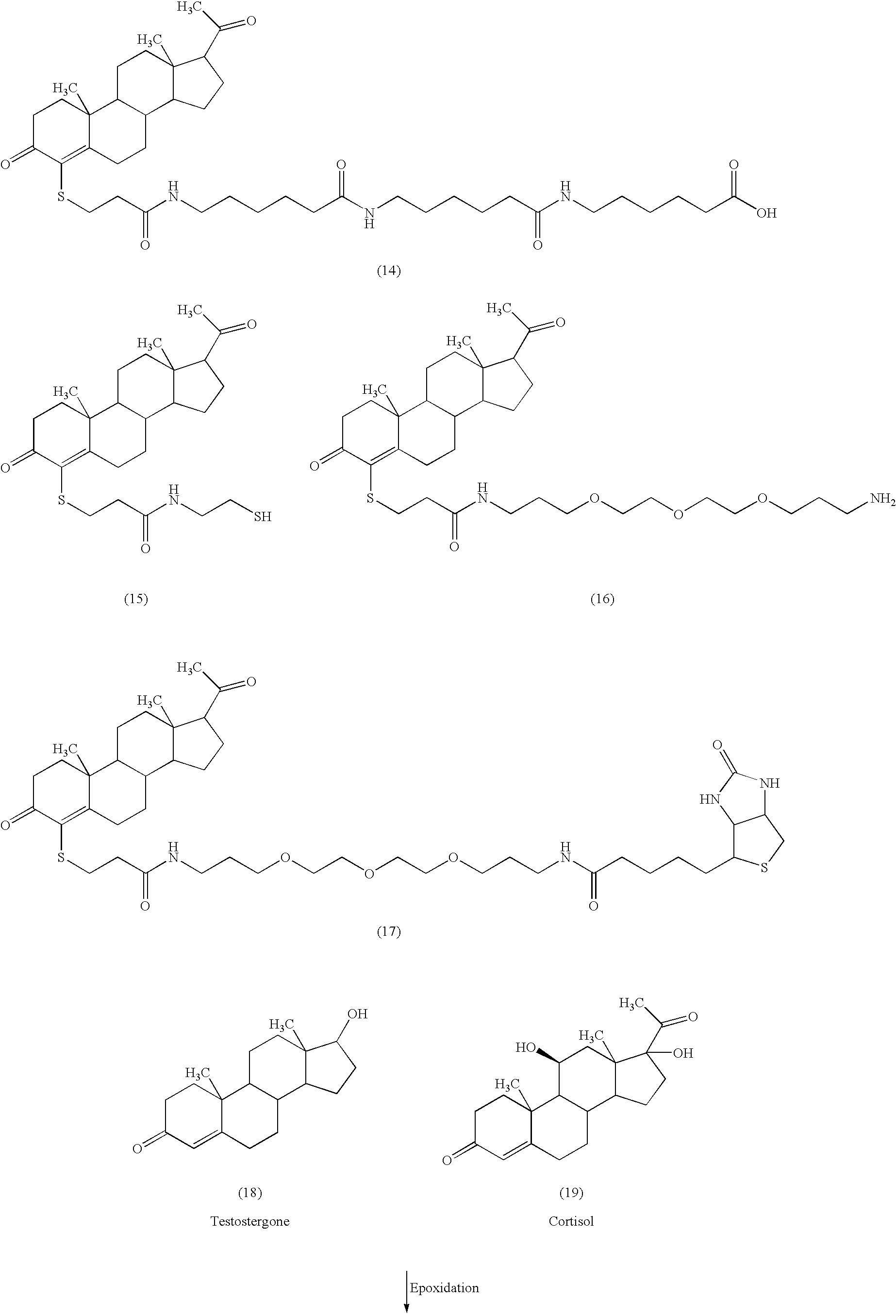

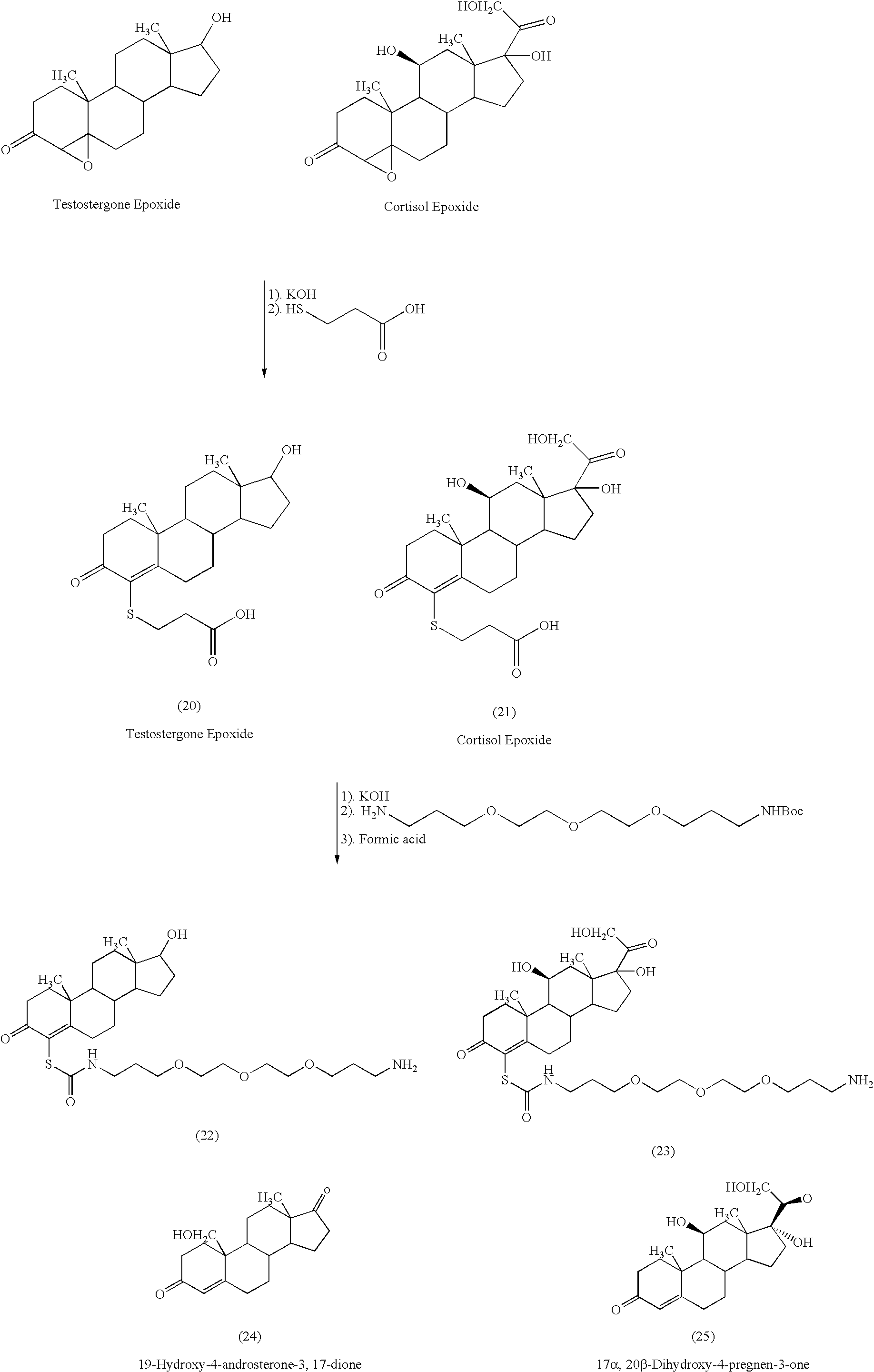

### Estrogens and Their 4-Position Functional Derivatives

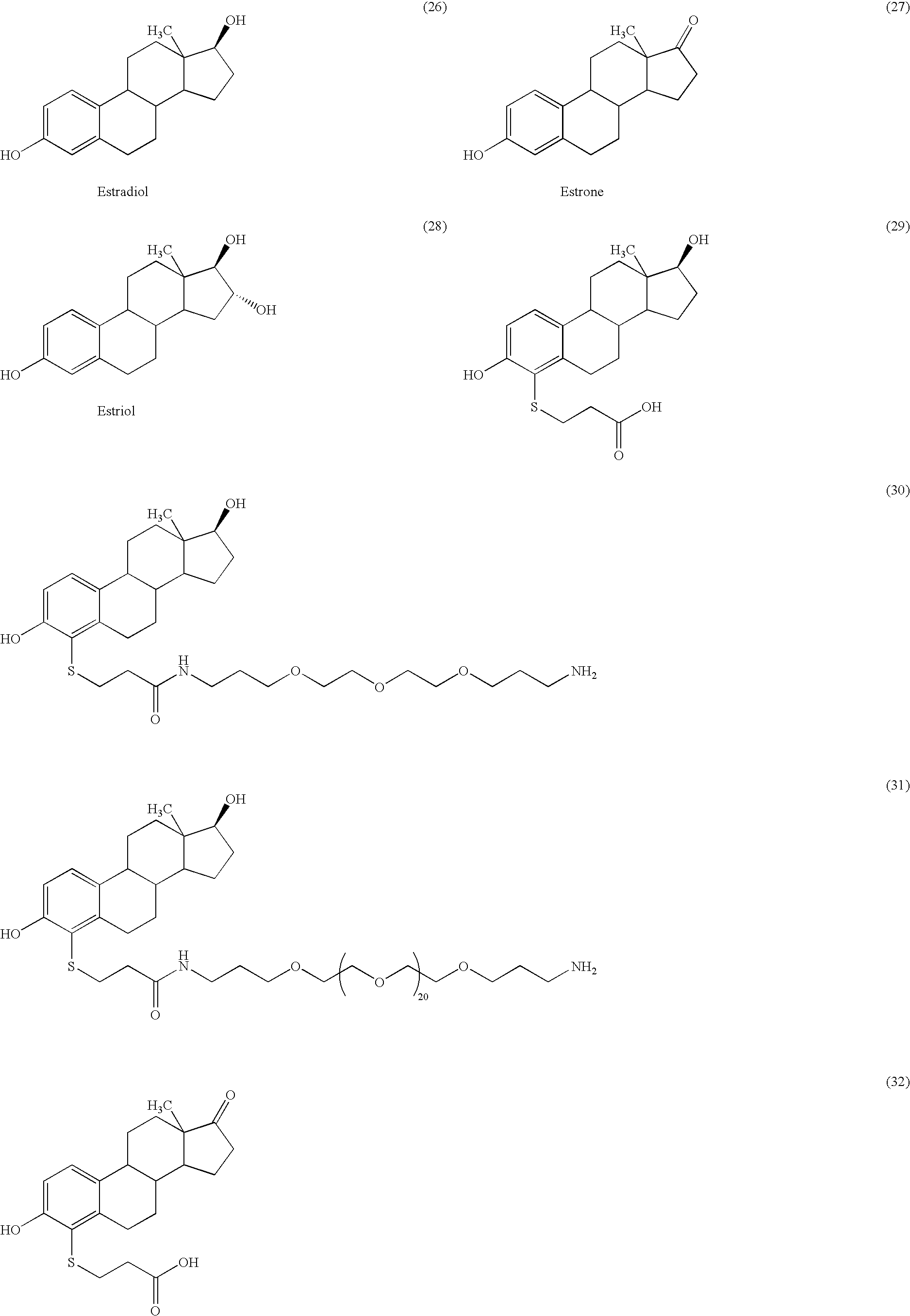

### Catecholamines and Their Aromatic Functional Derivatives

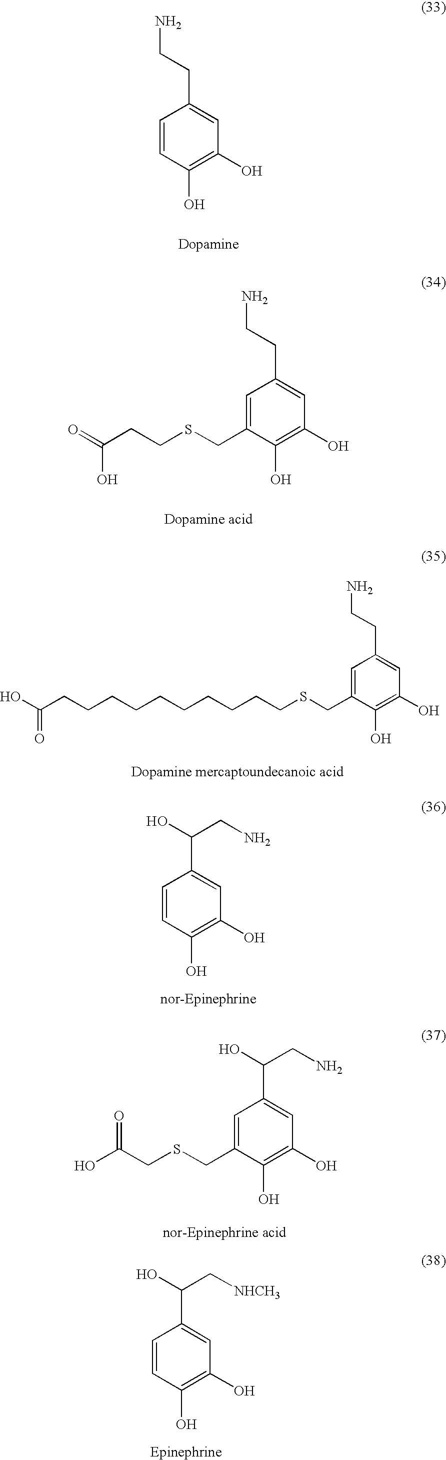

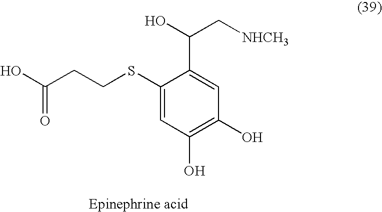

### Example 1

Synthesis of Progesterone-PEG-NH₂ Derivative (6, Reaction Scheme 2)

4-mercapto-progesterone acid (4) (200 mg) was dissolved in DMF (dry, 1 mL) and DCC (128 mg in 0.5 mL dry DMF) was added dropwise followed by NHS (71.3 mg in 0.5 mL dry DMF). The reaction was stirred in the dark overnight before filtering off the solid. Mono-PEG-Boc (458.2 mg) was dissolved in dry chloroform (1 mL) and added dropwise to the stirring ester solution. Triethylamine (0.5 mL) was then added and the reaction stirred over the weekend in the dark. The solvent was removed in vacuo and the mixture was separated by column using 15:1 chloroform:methanol eluent to yield yellow oil for amine-protected product (progesterone-PEG-NHBoc). Yield: 169.8 mg (49%). R_{f}=0.36 (15:1 chloroform:methanol). ¹H NMR (CDCl₃): δ: 0.65 (s, 3H, 18-CH₃), 1.13 (s, 3H, 19-CH₃), 1.41 (s, 9H, Boc CH₃), 2.09 (s, 3H, 21-CH₃), 2.89 (m, 6H, PEG), 3.57 (m, 14H, PEG). ¹³C NMR (CDCl₃) δ: 13.7 (18-CH₃), 18-4 (19-CH₃), 21.5 (11-CH₂), 23.2 (15-CH₂), 24.5 (16-CH₂), 25.3, 26.0 (S—CH₂), 28.7 (Boc CH₃), 29.4, 30.0, 30.9, 31.0 (7-CH₂), 31.7 (21-CH₃), 32.4 (6-CH₂), 34.3, 34.7 (1-CH₂), 34.9, 35.6, 36.7 (17-CH), 37.0, 38.9, 39.1 (12-CH₂), 41.7 (10-C), 44.2 (13-C), 49.1, 54.5 (9-CH), 56.3 (14-CH), 63.7 (17-CH), 69.8 (PEG C—O), 70.0 (PEG C—O), 70.5 (PEG C—O), 70.9 (PEG C—O), 129.0 (4-C), 156.3, 162.8, 171.4 (5-C), 176.2 (carbonyl), 195.7 (3-C), 209.5 (20-C). ES-MS (MeOH): [M+H]⁺ 722, [M+Na]⁺ 744.

The final free amine product or progesterone-PEG-NH₂ (6) can be easily synthesised from the above Boc-protected compound by deprotection in formic acid (98% pure).

### Example 2

Synthesis of Progesterone-PEG-Biotin (17)

Progesterone-PEG-NH₂ (6) (160 mg) was dissolved in chloroform (1.5 mL, dried over molecular sieves 4A). Biotin active ester (113.8 mg in 1 mL of dry DMF with warming) was added dropwise to the stirring progesterone-PEG-NH₂ solution. The solution was stirred in the dark for two hours before addition of triethylamine (0.5 mL) after which it was left stirring over the weekend. A solid initially forms but by the end of the reaction it has gone. The solvent was removed in vacuo and then column separated using 10:1 chloroform:methanol and 5:1 chloroform:methanol eluent. Yield (17): 95.5 mg (44%). R_{f}=0.70 (5:1 chloroform:methanol). ¹H NMR (CDCl₃): δ 0.70 (s, 3H, 18-CH₃), 1.25 (s, 3H, 19-CH₃), 1.72 (m, biotin), 1.80 (m, biotin), 2.14 (s, 3H, 21-CH₃), 2.95 (m, 5H, PEG), 3.20 (d, 1H, biotin), 3.37 (m, 2H, PEG), 3.62 (m, 13H, PEG), 4.36 and 4.54 (d of t, 2H, biotin), 5.16 and 5.23 (d, 1H, biotin). ¹³C NMR δ. ES-MS: 848.1 [M+H]⁺, 870.1 [M+Na]⁺.

### Example 3

Preparation of 4-Progesterone Acid Derivative (14) and its Ovalbumin Conjugate

A solution of ε-aminocaproic acid (44.4 mg (0.34 mM) in 200 μL of UHQ water) was added drop-wise to a solution of progesterone 18-atom linker-succinate active ester (Steroids, 67, 2002, 565-572) (83.8 mg (0.11 mM) in 2 mL of dry DMF). 0.5 mL of dry DMF was used to wash out the ε-aminocaproic acid vial. The reaction was stirred over a weekend. The solvent was removed under vacuum and the resultant yellow-tinged oil reconstituted in 100 mL of chloroform and washed with 3×50 mL of distilled water. The solvent was removed under vacuum, and the resultant light brown oil was column separated using a 15:1, 10:1, 5:1, 1:1, 0:1 chloroform:methanol eluent series. The resultant clear, colourless oil was washed with a diethyl ether, n-hexane, chloroform mixture to give waxy white solids (14). Yield: 68.1 mg (80%). R_{f}=0.77 (5:1 chloroform:methanol). ¹H NMR: δ 0.68 (s, 3H, 18-CH₃), 1.25 (s, 3H, 19-CH₃), 2.14 (s, 3H, 21-CH₃), 2.84 (t, 2H, J=6.8 Hz, S—CH₂), 3.71 (d of t, 1H, J=14.7 Hz, 6α-H). ¹³C NMR: δ 13.4 (18-C), 17.6, 18, 18.2 (19-C), 21.2 (11-C), 22.9 (15-C), 23.3, 24.3 (linker C), 25, 25.6, 26, 26.6, 29.2 (linker C), 29.8, 30.5, 30.8, 31.2, 31.8 (21-C), 31.9, 32.1 (6-C), 34.5, 34.7, 34.9, 35.7 (8-C), 36.8 (1-C), 36.9, 38.7, 39.6 (16-C), 39.8, 41.6 (10-C), 44 (13-C), 54.2 (9-C), 56, 63, 63.5 (17-C), 65.9, 171.5 (5-C), 173.8, 176.9, 196 (3-C), 209.5 (20-C), one overlapping peak. Analytical HPLC: 100% pure. 50° C., gradient of 30% B over 5 min. then 30-80% B over 25 min., A=90:10 dH₂O: MeOH, B=90:10 MeOH:dH₂O, PH_{A&B}=4.2, Rₜ=22.1 min. ES-MS: (MeOH, 40 V) 759 [M+H]⁺, 781 [M+Na]⁺.

A solution of DCC (17.7 mg in 250 μL dry DMF) was added drop-wise to a stirring solution of above progesterone acid derivative 14 (50 mg in 2 mL of dry DMF) and 250 μL of dry DMF used to wash out the vial. A solution of NHS (9.9 mg in 250 μL of dry DMF) was then added drop-wise and a further 250 μL of dry DMF used to wash. 0.5 mL of DMSO was then added to aid dissolution. The reaction was left stirring in the dark overnight. Conjugation to OVA was then done as the same procedure for other conjugates to produce conjugate (3) (Steroids, 67, 2002, 565-572).

### Example 4

Synthesis of progesterone-4-mercaptopropionamide-ethylthiol (15)

Progesterone-4-mercaptopropionyl succinate (Steroids, 67, 2002, 565-572) (100 mg, 0.194 mmol) was dissolved in dry DMF (1 mL) and a solution of mercaptoethylamine (44.8 mg, 0.581 mmol, in 0.5 mL dry DMF) was added drop-wise followed by a further 0.5 mL of DMF to wash. The reaction was stirred overnight at room temperature. Solid formed was filtered off and the filtrate solvent was removed in vacuo. The resulting oil was washed with chloroform and the chloroform phase was column separated using CHCl₃, 15:1 CHCl₃:MeOH, 10:1 CHCl₃:MeOH, 5:1 CHCl₃:MeOH eluent to yield an oil. Yield: 17.1 mg (18%). R_{f}=0.52 (15:1 chloroform:methanol). ¹H NMR (CDCl₃): δ 0.70 (s, 3H, 18-CH₃), 1.26 (s, 3H, 19-CH₃), 2.15 (s, 3H, 21-CH₃), 2.45 (t, 1H, J=7 Hz), 2.53 (m, 3H), 2.88 (m, 4H, 2×S—CH₂), 3.62 (m, 2H, CONH—CH₂), 3.73 (d, 1H, J=14 Hz, 6α-H). ¹³C NMR (CDCl₃): δ 13.4 (18-C), 18.1 (19-C), 20.8 (11-C), 23.0 (15-C), 24.3 (16-C), 25.0 (S—CH₂), 25.7 (S—CH₂), 30.5 (7-C), 31.5 (21-C), 32.1 (C-6), 34.0 (2-C), 34.2 (N—CH₂), 34.4 (1-C), 35.7 (8-C), 36.5 (CH₂CO), 38.7 (12-C), 41.6 (10-C), 43.8 (13-C), 54.2 (9-C), 55.8 (14-C), 63.5 (17-C), 129 (4-C), 172 (5-C), 175 (amide C═O), 195 (3-C), 209 (20-C). ES-MS: 476 Da [M−H]⁻.

### Example 5

Synthesis of Testosterone-PEG-NH₂ Derivative (22)

Testosterone (18) (807.5 mg, 2.8 mmol) was dissolved in methanol (45 ml). The solution was stirred and cooled to 0° C. on ice, after which 10% w/v sodium hydroxide was added (3.4 ml in distilled water), followed by 30% hydrogen peroxide (3.7 ml). The reaction was then stirred at 0° C. for four hours. The reaction solution was then raised to room temperature and the pH adjusted to 7.0 with 2 M acetic acid and the solvent removed in vacuo before drying. The resulting clear, colourless semi-solid was partially dissolved in distilled water (30 ml) and then extracted with ethyl acetate (3×30 ml). The organic phase was then washed with distilled water (1×30 ml) and dried over sodium sulphate. The solution was then decanted and the solvent removed and the sample dried to yield testosterone epoxide as a tacky solid. Yield: 810.0 mg (96%). R_{f}=unknown (no UV absorbance). IR (neat): 1055, 2362, 2945, 3584 cm⁻¹. ¹H NMR: (CDCl₃) δ 0.76 (3H, s, 18-CH₃), 1.17 (3H, s, 19-CH₃), 2.98 (1H, s, 4-H), 3.4-3.7 (6ε- and 17α-H). ¹³C NMR: δ 11.1 (18-CH₃), 19.3 (19-CH₃), 21.1 (CH₂), 23.1 (CH₂), 26.1 (CH₂), 29.9 (CH₂), 33.1 (CH₂), 35.1 (CH), 36.5 (CH₂), 38.0 (CH₂), 43.0 (C), 46.6 (CH), 50.4 (CH), 60.7 (C), 62.6 (CH), 70.5 (C), 81.3 (CH), 207.5 (3-carbonyl). ES-MS: (MeOH, −20V): 353.1 [M+MeOH+H₂O—H]—. Mp=100-102° C. Lit mp: 156-157° C. HPLC: 60% MeOH, 100% purity, Rₜ=9.83 min. λₘₐₓ=203 nm.

Testosterone epoxide (517.5 mg, 1.7 mmol) was dissolved in ethanol (5 ml, dried over molecular sieves). In a 20 ml flask, 25% w/v potassium hydroxide (0.8 ml in distilled water) was added with 3-mercaptopropionic acid (244 μl, 2.8 mmol). The epoxide solution was then added slowly to the stirring MPA solution and the sample immediately placed under nitrogen and stirred for four hours. Distilled water (30 ml) was then added which immediately precipitated a white solid. The sample was then extracted with diethyl ether (3×30 ml) and the aqueous phase was pH adjusted to 1.5 with 1M HCl and further extracted with ethyl acetate (3×30 ml). The combined organic phase was then dried over sodium sulphate and the solvent removed and the sample dried to yield testosterone acid (20) as a white solid. Yield: 642.3 mg (96%). R_{f}=0.25 (15:1 chloroform:methanol). IR (neat): 1708, 2288, 2935 cm⁻¹. ¹H NMR: δ 0.77 (18-CH₃), 1.16 (19-CH₃), 2.52 and 2.69 (1H each, t, J=7.3 Hz, CH₂—COOH), 2.78 and 2.99 (1H each, m, CH₂—S), 3.67 (1H, t, J=11.2 Hz, 6α-H), 4.12 (1H, q, J=9.5 Hz, 17α-H). ¹³C NMR: δ 11.1 (18-CH₃), 19.0 (19-CH₃), 19.6 (CH₂), 23.6, 26.0 (S—CH₂), 29.8 (CH₂), 29.9 (16-CH₂), 32.4 (12-CH₂), 35.0 (8-CH), 36.2 (1-CH₂), 37.2 (C), 38.2 (CH₂), 42.9 (C), 46.5 (CH), 50.6 (CH), 61.0 (CH₂), 62.6 (CH), 70.4 (C), 81.5 (CH), 175.7 (acid), 207.2 (3-carbonyl). ES-MS (40V, MeOH): 393.3 [M+H]⁺, 415.0 [M+Na]⁺. Mp=112-116° C./132-136° C. Lit mp: 156-159/179-181° C. HPLC: 60% methanol, Rₜ=4.47 min., % Purity=96%.

Testosterone acid (20) (642.3 mg, 1.637 mmol) was dissolved in dry DMF (5 ml, dried over molecular sieves). DCC (416.4 mg, 2.128 mmol, in 1 ml dry DMF) was added dropwise to the stirring steroid solution, followed by NHS (232.1 mg, 2.128 mmol, in 1 ml of dry DMF) was also added dropwise. The solution was stirred at room temperature for 48 hours in the dark. The white solid formed was filtered off and washed thoroughly with dry DMF. The filtrate had solvent removed and sample dried in vacuo. The sample was then column separated using chloroform and 15:1 chloroform:methanol as eluent yielding testosterone succinimide ester as a white semi-solid. Yield: 783.0 mg (98%). R_{f}=0.41 (15:1 chloroform:methanol). IR (neat): 1207, 1630, 1737, 2931 cm⁻¹. ¹H NMR: δ 0.76 (3H, s, 18-CH₃), 1.16 (3H, s, 19-CH₃), 2.85 (4H, s, NHS protons), 3.64 (2H, m, 6α-H and 17α-H). ¹³C NMR: δ 11.1 (18-CH₃), 18.9 (19-CH₃), 21.1 (CH₂), 23.4 (CH₂), 25.1 (CH₂) 25.6 (NHS CH₂), 29.6 (CH₂), 29.9 (CH₂), 32.4 (CH₂), 35.1 (CH), 36.4 (CH₂), 37.2 (C), 41.5 (CH₂), 43.0 (C), 46.5 (CH), 50.7 (CH), 54.4 (CH₂), 62.6 (CH), 70.3 (C), 81.5 (CH), 167.1 (amide), 169.2 (NHS carbonyl), 207.2 (3-carbonyl). ES-MS: (MeOH 40V): 490.3 [M+H]⁺. Lit mp: 154-156° C. HPLC: 5% MeOH, Rₜ=2.03 min, λₘₐₓ=259 nm, % purity=100%.

Testosterone succinimide ester (658.9 mg, 1.347 mmol) was dissolved in dry DMF (3.5 ml) and stirred whilst a solution of mono-Boc-PEG was added dropwise (646.2 mg, 2.021 mmol, in 1.5 ml of dry chloroform) followed by a chloroform rinse (250 μl). Triethylamine (750 μl) was then added to the stirring solution and the solution stirred at room temperature in the dark for 60 hours. The solvent was then removed and sample dried in vacuo and the sample column separated using chloroform, 15:1 chloroform:methanol and 10:1 chloroform:methanol as eluent to yield testosterone-PEG-Boc as an orange oil. Yield 724.5 mg (77%). R_{f}=0.50 (10:1 CHCl₃: MeOH). IR (neat) 1532, 1659, 2931, 3335 cm⁻¹. ¹H NMR: δ 0.80 (3H, s, 18-CH₃), 1.24 (3H, s, 19-CH₃), 1.43 (9H, s, Boc methyls), 1.77 (4H, m, O—CH₂—CH₂—CH₂—NH), 2.58 (2H, t, J=7.1 Hz, CH₂—CONH), 2.96 (2H, t, J=7.7 Hz, S—CH₂), 3.20 (2H, d of t, J_{d}=6.7 Hz, CH₂—CO—NH—CH₂), 3.41 (2H, d of t, J_{d}=5.9 Hz, Jₜ=5.8 Hz CH₂—NH—CO), 3.52-3.66 (12H, m, O—CH₂). ¹³C NMR: δ 11.1 (18-CH₃), 18.9 (19-CH₃), 21.1 (CH₂), 23.4 (CH₂), 25.1 (CH₂), 28.4 and 28.9 (O—CH₂—CH₂—CH₂—NH) 29.0 (CH₂), 29.6 (S—CH₂), 30.1 (CH₂), 34.1 and 35.7 (CH₂—CO—NH—CH₂), 35.3 (CH), 36.5 (CH₂), 37.6 (C), 38.3 (C), 42.8 (CH₂), 46.5 (CH), 50.4 (CH), 54.4 (CH₂), 63.0 (CH), 70.1 (cluster, CH₂—O) 70.4 (C), 81.1 (17-CH), 156.1 (Boc terminal amide), 168.8 (steroid terminal amide), 195.6 (3-carbonyl). ES-MS: (MeOH 40V): 695.6 [M+H]⁺, 717.6 [M+Na]⁺, 815.5 [M+2CH₃COOH+H]⁺, 837.5 [M+2CH₃COOH+Na]⁺. Analytical HPLC: MeOH mobile phase, 1 ml/min. 95% pure, Rₜ=2.03 min, 206 nm.

The final free amine product or testosterone-PEG-NH₂ (22) can be easily synthesised from the above Boc-protected compound by deprotection in formic acid (98% pure).

### Example 6

Synthesis of Cortisol-PEG-NH₂ Derivative (23)

Cortisol (19) (362.5 mg, 1.0 mmol) was partially dissolved in methanol (13 ml) and ethanol (5 ml) and chilled to 0° C. Sodium hydroxide solution (10% w/v in distilled water, 1 ml) was added followed by 30% hydrogen peroxide solution (400 μl). The reaction was kept stirring at 0° C. on ice for three hours. The reaction mixture was then raised to room temperature; any remaining solid was filtered off using a sintered glass funnel. The filtrate pH was carefully adjusted to 7.0 using acetic acid and the resulting solution dried in vacuo to yield a clear, colourless oil. This sample was then constituted in distilled water (30 ml) and extracted with 3×30 ml of ethyl acetate.

The organic phase was then washed with 1×30 ml of distilled water and the organic phase dried over sodium sulphate. The supernatant was then passed through a bed of calcined alumina (˜10 g) and the solvent removed and sample dried in vacuo to yield cortisol epoxide as clear, colourless oil. The product was then column separated using 1:1 ethyl acetate:n-hexane to yield as an analytical sample. Yield: 86.6 mg (23%). R_{f}=0.36 (1:1 ethyl acetate:n-hexane). IR (KBr disc): 1450, 1701, 1724, 2369, 2928, 3449 cm⁻¹. ¹H NMR (δ): 1.14 (3H, s, 18-CH₃), 1.36 (3H, s, 19-CH₃), 3.03 and 3.06 (1H, s, 4-H, β and α respectively), 4.30, 4.40 (1H each, d, J=3.7 Hz, 21-H). ¹³C NMR (δ): 15.9 (18-CH₃), 20.0, 21.1, 22.2, 25.8, 28.3, 28.6, 29.0, 29.4, 30.4 (19-CH₃), 32.9, 35.2, 35.3, 40.6, 52.2, 62.8, 62.9, 68.0, 68.6, 206.5, 218.9. ESMS (−40V, MeOH): 363.2 [M+H₂O—H]⁺. Melting point: 157-160° C. β epimer. 166-169° C. α epimer. Lit. Mp: β 147-148° C., α 167-168° C. HPLC: 1 ml/min. 60% MeOH, 100% purity, Rₜ=4.60 and 4.85 min for the two epimers, λₘₐₓ=204 nm.

Cortisol epoxide (586.8 mg, 1.559 mmol) was dissolved in ethanol (dried over molecular sieves, 5 ml). A solution of potassium hydroxide (25% w/v in distilled water, 730 μl) was added to a small flask and stirred whilst 3-mercaptopropionic acid (224 μl) was added. The stirring solution then had the epoxide solution added dropwise and was immediately placed under nitrogen and stirred at room temperature for four hours. Distilled water (30 ml) was added. The aqueous phase was then extracted with diethyl ether (3×30 ml) before adjusting the pH of the aqueous phase to 1.5 with 1M HCl. The aqueous phase was then extracted with 3×30 ml of ethyl acetate. The organic phase was then dried over sodium sulphate and the liquor decanted and solvent removed and sample dried in vacuo. The sample was then column separated using chloroform, 15:1 chloroform:methanol and methanol eluent. The sample was then dried to yield 4-mercapto-cortisol acid (21) as clear, colourless oil. Yield: 479.9 mg (66%). R_{f}=0.42 (5:1 chloroform:methanol). IR (neat): 1108, 1657, 2360, and 2920. ¹H NMR: δ 0.89 (3H, s, 18-CH₃), 1.21 (1H, t, J=7.0 Hz), 1.47 (3H, s, 19-CH₃), 2.47 (2H, t, J=7.0 Hz, CH₂—COOH), 2.84 (2H, t, J=7.1 Hz, S—CH₂), 3.66 (1H, q, J=7.0 Hz), 4.28 (1H, d, J=19.4 Hz, 21-H), 4.66 (1H, d, J=19.4 Hz, 21-H). ¹³C NMR: δ, 21.4, 22.1, 26.0, 26.2 (S—CH₂), 33.1 (19-CH₃), 35.4, 38.1, 38.4, 39.5, 46.3, 51.7, 53.3, 54.1, 56.1, 60.2, 71.1, 72.1, 93.2, 130.5, 179.6 (carboxylic acid), 182.9 (17-C), 200.8 (20-carbonyl), 216.9 (3-carbonyl). ES-MS (40V, MeOH): 466.1 [M+H]⁺, 488.0 [M+Na]⁺. Mp: 132-136° C. Lit. Mp: 177-178° C. HPLC: 1 ml/min. 60% v/v methanol. Rₜ=1.95 min. % Purity=100%.

Cortisol acid (21) (479.9 mg, 1.029 mmol) was dissolved in dry DMF (4 ml, dried over molecular sieves) and DCC (275.9 mg, 1.337 mmol, in 1 ml dry DMF) was added dropwise to the stirring steroid solution. This was followed by NHS (153.9 mg, 1.337 mmol, in 1 ml dry DMF) dropwisely. The reaction was stirred overnight at room temperature in the dark. The white solid formed was then filtered off and washed with dry DMF and the filtrate solvent removed in vacuo. The sample was then column separated using chloroform, 15:1 chloroform:methanol, 10:1 chloroform:methanol to yield cortisol succinimide ester as a pale yellow semi-solid. Yield: 486.9 mg (84%). R_{f}=0.69 (5:1 chloroform:methanol). IR (KBr disc): 1078, 1655, 1736, 2928 cm⁻¹. ¹H NMR: δ 0.90 (3H, s, 18-CH₃), 1.50 (19-CH₃), 2.64 (2H, t, J=6.8 Hz), 2.83 (2H, t, J=6.5 Hz), 2.88 (4H, d, J=1.2 Hz, NHS protons), 4.29 (1H, s, broad, 21-H). ¹³C NMR: δ 16.9 (18-CH₃), 21.8, 23.8, 25.1, 25.8 (S—CH₂), 28.1, 30.6, 31.9, 33.1 (19-CH₃), 33.7, 34.0, 34.4, 39.4, 42.3, 47.7, 48.7, 52.0, 56.4, 68.0, 89.6, 125.6, 158.4, 167.7, 171.0, 179.6 (17-C), 196.4 (20-carbonyl), 206.8 (3-carbonyl). ES-MS: (40V, MeOH) 695.7 [M+DMF+2H₂O+Na]⁺. Mp: 139-142° C. HPLC: 30% methanol, Rₜ=1.86 min, % Purity=90%.

Cortisol succinimide ester (486.9 mg, 0.864 mmol) was dissolved in dry DMF (3.5 ml, dried over molecular sieves). To the stirring steroid solution, was added mono-Boc PEG (416.0 mg, 1.296 mmol, in 1.2 5 ml of dry chloroform (dried over molecular sieves) dropwise, with an additional 2×250 μl of dry chloroform used to wash. The stirring solution had dry triethylamine added (750 μl, dried over molecular sieves). The reaction was then stirred at room temperature in the dark for 60 hours. After 12 hours, another 1 ml of dry DMF was added to aid solubility. The reaction was then stopped and solvent removed and sample dried in vacuo before column separation using chloroform, 15:1 chloroform:methanol and 10:1 chloroform:methanol as eluent, yielding cortisol-PEG-Boc compound as an orange oily solid. Yield: 413.6 mg (62%). R_{f}=0.32 (10:1 chloroform:methanol). IR (KBr disc) 1707, 2930, 3437 cm⁻¹. ¹H NMR: δ 0.90 (3H, s, 18-CH₃), 1.43 (9H, s, Boc methyls), 1.50 (3H, s, 19-CH₃), 1.71-1.78 (6H, m, 4H from O—CH₂—CH₂—CH₂—NH, 2H from steroid fine structure), 2.60 (2H, m, CH₂—COOH), 2.82 (2H, m, CH₂—S), 3.11 (2H, t, J=6.6 Hz, CH₂—CO—NH—CH₂), 3.26 (2H, m, CH₂—NH—CO), 3.50-3.70 (14H, m, 12H from O—CH₂, 2H from steroid fine structure). ¹³C NMR: δ 16.8 (18-CH₃), 21.5, 22.0, 25.6, 27.7, 27.9, 28.1, 28.3 and 28.6 (O—CH₂—CH₂—CH₂—NH), 29.5 (S—CH₂), 29.8 (CH₂), 30.3, 33.8 (19-CH₃), 34.5, 35.0, 37.9 (C), 42.4 (CH₂), 47.9, 48.1, 48.4, 48.6, 52.2, 52.4, 56.7, 69.0, 69.1, 69.8, 70.1 and 70.3 and 70.6 (CH₂—O), 79.0, 89.6, 126.1, 126.4, 157.3 (Boc terminal amide), 172.7 (steroid terminal amide), 178.9, 196.5 (3-carbonyl), 206.0 (20-carbonyl). ES-MS: m/z (MeOH, 40V) 385.4 [M+2H]²⁺. Mp: 32-33° C. HPLC: Purity: 99%. MeOH mobile phase, 1 ml/min. Rₜ=1.92 min, λₘₐₓ=206 nm.

The final free amine product or cortisol-PEG-NH₂ (23) can be easily synthesised from the above Boc-protected compound by deprotection in formic acid (98% pure).

### Example 7

4-Mercaptol-Estradiol Acid (29)

4-bromoestradiol (200 mg) was dissolved in dry methanol (20 mL). Methanolic potassium hydroxide (20 mL, 7.8 mgmL⁻¹) was added followed by 3-mercapto-propionic acid (550 μL). The solution was refluxed under dry conditions for 24 hours in the dark. The solvent was removed and the sample reconstituted in distilled water (50 mL). The aqueous phase was washed with ethyl acetate (2×25 mL, 1×50 mL). The aqueous phase had its pH adjusted to 2.5, which crashed a white solid out of solution. The solid was separated by centrifugation and washed three times with water and then dried to yield a white solid 29 (103.4 mg, 46%). mp 78-84° C.; R_{f}=0.46 (ethyl acetate); ¹H NMR 0.81 (3H, s, 18-CH₃), 1.38-2.3 (m, estradiol fine structure), 2.75 (3H, t, J=4.6, 17-CH), 2.81 (2H, t, J=4.5, S—CH₂), 6.89 (1H, d, J=6.3, 2-H), 7.22 (1H, d, J=6.7 Hz, 3-H); ¹³C NMR 10.4 (18-CH₃), 14.2, 21.2, 21.4, 22.8, 23.1, 24.0, 25.4, 26.8, 29.1 (S—CH₂), 29.8, 30.2, 31.0, 33.8, 34.2, 37.1, 50.9 (17-CH), 74.6, 90.5, 171.5 (3-C), 194 (COOH); ES-MS m/z 399.1 [M+H]⁺, 406.8 [M+OMe]⁻.

### Example 8

4-Estradiol-PEG-NH₂ (30)

4-Estradiol acid (29) (80 mg, 0.201 mmol) was dissolved in dry DMF (1 mL) and DCC (53.9 mg in 0.5 mL of dry DMF, 0.2613 mmol) was added dropwise to the vigorously stirring solution followed by NHS (30.1 mg in 0.5 mL of dry DMF, 0.2613 mmol). The solution was stirred overnight at room temperature in the dark. A white solid formed within 30 min of addition. The solid was filtered off and the solvent removed. The sample was then column separated using 15:1 chloroform:methanol, 10:1 chloroform:methanol and 5:1 chloroform:methanol. The pure product (4-estradiol succinimidyl ester) was isolated as a white solid (44.0 mg, 44%). Mp=149-156° C. R_{f}=0.48 (10:1 chloroform methanol). ¹H NMR: δ 0.82 (3H, s, 18-CH₃), 1.05-2 (m, estradiol fine structure), 2.73 (t, 17 CH), 2.90 (2H, t), 2.97 (4H, s, NHS protons), 8.03 (2H, s, aromatic ring); ¹³C NMR 25.2 (CH₂), 25.7 (CH₂), 25.9 (CH₂), 27.3 (CH₂), 29.9 (S—CH₂), 30.0 (CH₂), 31.5 (18-CH₃), 31.9 (CH₂), 32.7 (CH₂), 33.5 (CH₂), 34.0 (CH₂), 34.3 (CH₂), 34.5 (succinate CO), 35.0 (succinate CO), 37.0 (CH), 49.8 (CH), 51.0 (17-CH), 52.2 (CH), 154.1 (C), 158.0 (C), 163.3 (CH), 169.2 (C), 172.5 (CH), 175.2 (3-C), 175.4 (ester); ES-MS m/z 471.6 [M+H]⁺.

The above synthesised 4-estradiol succinimidyl ester (50 mg, 0.106 mmol) was dissolved in dry DMF (1 mL) and stirred rapidly whilst mono-Boc protected PEG (220) (102.6 mg, 0.372 mmol in chloroform, 0.5 mL) was added drop-wise followed by triethylamine (0.25 mL). The solution was then stirred over the weekend at room temperature in the dark. The solvent was then removed and the resulting oil column separated using 15:1 chloroform:methanol, 10:1 chloroform:methanol, 5:1 chloroform:methanol eluent sequence, yielding pure compound [4-estradiol-PEG (220)-NHBoc] as a clear, colourless oil (62.3 mg, 0.098 mmol, 93% yield). R_{f}=0.36 (10:1 chloroform:methanol). ¹H NMR: δ 1.24 (2H, t, J=7.0), 1.44 (9H, s, Boc CH₃), 1.79 (5H, m), 2.59 (2H, t, J=7.4), 2.74 (3H, t, J=6.2), 2.98 (5H, m), 3.37 (2H, m), 3.60 (14H, m), 5.06 (1H, s), 6.82 (1H, s, aromatic estradiol); ¹³C NMR: 18.4 (estradiol CH₃), 26.4, 27.2, 28.5, 28.7 (Boc CH₃), 29.7, 33.2, 33.3, 33.8, 34.0, 34.3, 34.6, 36.2, 36.5, 38.0, 38.4, 50.6, 52.0, 58.4, 69.4, 69.9 (PEG C—O) 70.1 (PEG C—O), 70.2 (PEG C—O), 70.5 (PEG C—O), 70.5 (PEG C—O), 79.3 (17-CH), 100.3, 102.8, 109.8, 127.6, 139.1, 156.3, 171.4 (CH), 171.7, 175.1 (Boc carbonyl), 181.1 (mercaptol-propionate carbonyl); ES-MS (MeOH, 45V) 535.4 [M-Boc+H]⁺, 557.4 [M-Boc+Na]⁺, 652.4 [M+NH₄]⁺, 670.4 [M+H₂O+NH₄]⁺.

The final free amine product or 4-estradiol-PEG-NH₂ (30) can be easily synthesised from the above Boc-protected compound by deprotection in formic acid (98% pure).

### Example 9

4-Estradiol-PEG (900)-NH₂ (31)

Polyethylene glycol (900) [O,O′-Bis-(2-aminopropyl)polypropylene glycol-block-polyethylene glycol-block polypropylene glycol, Fluka 14527] (2 g, approx. 2.22 mmol) was dissolved in dry methanol (20 mL) and dry triethylamine (1 mL) was then added. Boc reagent (0.4856 g, 2.22 mmol) was dissolved in dry methanol (10 mL) and added drop-wise to the above rapidly stirring PEG solution over ˜20 min using a syringe and septum. The solution was then left to rapidly stir overnight at room temperature. The solvent was then removed and the sample was separated by a column using 32:1:1, 32:2:1, 32:4:1, 16:4:1 dichloromethane:methanol:acetic acid eluent to yield mono-protected PEG (900) as a clear colourless semi-solid (911.4 mg, 41% yield). R_{f}=0.53 (32:2:1 dichloromethane:methanol:acetic acid). ¹H NMR: δ 1.13 (s, 8H), 1.27 (s, 3H), 1.44 (s, 9H, Boc CH₃), 2.00 (s, 6H), 3.45 (s, 7H), 3.65 (s, 65H, ethylene protons); ¹³C NMR: 15.0, 15.3, 15.4, 16.1, 16.8, 16.9, 17.0, 17.9, 18.8, 22.5, 28.4 (Boc CH₃), 46.6, 47.1, 47.2, 48.4, 70.3 (cluster), 72.5, 72.6, 74.4, 74.9, 75.2, 75.5, 76.2, 155.5, 176.1 (Boc-carbonyl). ES-MS: (MeOH 40V) multiple peaks corresponding to different n-values of the PEG chain.

4-Estradiol succinimidyl ester (50 mg, 0.106 mmol) was dissolved in dry DMF (1 mL) and stirred rapidly whilst mono-Boc PEG (900) (371.7 mg, approx. 0.372 mmol dissolved in 5:1 chloroform:methanol, 3 mL) was added drop-wise followed by triethylamine (0.5 mL). The solution was stirred at room temperature over the weekend in the dark. The solvent was then removed and the resulting orange oil column separated using 15:1 chloroform:methanol, 10:1 chloroform:methanol, 5:1 chloroform:methanol eluent to yield pure protected product [4-estradiol-PEG (900)—NHBoc] as a clear, colourless oil (39.5 mg, 0.029 mmol, 27% yield). R_{f}=0.73 (5:1 chloroform:methanol). ¹H NMR: δ 1.14 (14H, m), 1.44 (9H, s, Boc CH₃), 2.58 (2H, t, J=7.1), 2.73 (3H, t, J=7.0), 2.97 (6H, m), 3.47 (m), 4.91 (1H, s), 6.75 (1H, t of d, J=34.9, J=7.9); ¹³C NMR: 16.7, 17.1, 17.6, 18.0, 28.5 (Boc CH₃), 29.7, 34.1, 34.3, 36.2, 45.1, 45.5, 70.6 (PEG C—O), 71.9 (PEG C—O), 72.1, 72.4, 72.6, 73.4, 74.0, 74.5, 75.1, 75.3, 75.6, 75.9, 126, 128, 130, 155.7, 164, 170.8, 174.4. ES-MS: (MeOH, 40V) multiple peaks from range of PEG chain n-values.

The synthesis of final 4-estradiol-PEG (900)-NH₂ (31) is carried out in the same procedure as for 4-estradiol-PEG-NH₂ (30) in formic acid (98% pure).

### Example 10

4-Mercapto-Estrone Acid (32)

Estrone (27) (400 mg, 1.48 mmol) was dissolved in dry ethanol (10 mL) and acetone (10 mL). N-bromosuccinimide (263.3 mg, 1.48 mmol) was added to the vigorously stirring solution and the solution stirred at room temperature for 24 hours. The white solid formed was filtered off and washed with ethanol (174.5 mg, 34%). Removal of the filtrate solvent and recrystalisation of the resultant solid as 4-bromoestrone provided 43% of yield. Mp 254° C. (literature 281-282° C.); R_{f}=0.23 (4:1 petroleum spirit 60-80° C.: ethyl acetate); ¹H NMR 0.90 (3H, s), 0.90 (1H, s), 1.26-2.96 (m), 5.37 (1H, s), 6.86 (1H, d, J=8.6 Hz), 7.18 (1H, d, J=8.6 Hz); ES-MS m/z.

4-bromoestrone (150 mg, 0.43 mmol) was dissolved in dry methanol (20 mL) and potassium hydroxide (15 mL, 23.4 mgmL⁻¹ in dry methanol) was added whilst stirring, followed by 3-mercaptopropionic acid (424.8 μL) and refluxed under dry conditions for 24 hours. The sample was then cooled and solvent removed. The sample was reconstituted in distilled water (25 mL) and extracted with ethyl acetate (2×12.5 mL, 1×25 mL). The solvent was removed and the sample recrystallized from chloroform to provide pure 4mercapto-estrone acid (32) (42.6 mg, 27%): Mp 108-112° C.; R_{f}=0.12 (15:1 chloroform:methanol); ¹H NMR 0.87 (3H, s, 18-CH₃), 1.23-3 (17H, m, estrone fine structure), 3.04 (2H, t, J=1.9, S—CH₂), 6.50 (1H, d J=8.7, C-2), 6.80 (1H, d, J=9.0, C-1); ¹³C NMR 17.5, 23.4, 25.5, 28, 30, 30.7, 34.5, 35, 39.5, 41.5, 42.3, 48.1, 54.2, 117, 118.8, 119.2, 123.5, 125.4, 129, 159, 178.4; ES-MS: m/z 374.5 [M+H]⁺, 397.5 [M+Na]⁺.

### Example 11

Dopamine 5-Mercaptopropanoic Acid (34)

Dopamine (33) (400 mg, 2.12 mmol) was dissolved in dry methanol (30 mL) and N-hydroxysuccinimide (375.2 mg, 2.12 mmol) was added and the solution stirred at room temperature in the dark for 24 hours. The solution then had the solvent removed and was reconstituted in distilled water (50 mL) and washed with chloroform (2×25 mL, 1×50 mL) and the solvent removed from the aqueous phase. The sample was reconstituted in methanol and decoloured thoroughly with activated charcoal. The solvent was then removed to yield 5-bromo-dopamine as an off-white semi-solid (239.5 mg, 49%). R_{f}=0.54 (40:1 methanol:acetic acid), ¹H NMR 2.94 (2H, t, J=7.2 NH₂—CH₂), 3.17 (2H, t, J=6.9 Ar—CH₂), 6.74 (1H, m, 2-CH), 6.92 (1H, m, 5-CH); ¹³C NMR 31.0 (Ar—C), 31.85 (Ar—C), 39.4 (C—NH₂), 40.5 (C—NH₂), 115.7 (2-C), 116.5 (5-C), 116.6 (6-C), 117.0 (3-C), 118.0 (4-C), 124.2 (1-C); ES-MS m/z 233 isotope pattern [M+H]⁺.

The above synthesised 5-bromo-dopamine (100 mg, 0.429 mmol) was dissolved in dry methanol (5 mL) and methanolic KOH was added (11.8 mgmL⁻¹, 5 mL) with vigorous stirring. 3-Mercaptopropionic acid (113.7 μL) was added and the reaction refluxed under dry conditions for 24 hours. The solvent was then removed and the resultant semi-solid constituted in distilled water (25 mL). The aqueous phase was washed with ethyl acetate (2×12.5 mL, 1×25 mL) and the aqueous phase acidified to pH=1. The solvent was removed from the aqueous phase to yield a yellow-white semi-solid (250.6 mg), which was then passed through a short silica column using 40:1 methanol:acetic acid eluent to yield pure product 34 as a white solid (44.1 mg, 40% yield). Mp=292-298° C., R_{f}=0.55 (40:1 methanol:acetic acid), ¹H NMR: δ 2.44 (2H, t, J=9.5, CH₂—COOH), 2.77 (2H, t, J=9.7, CH₂—S), 2.54-2.88 (2H, m, CH₂—Ar), 3.19-3.57 (m, CH₂—NH₂); ¹³C NMR: 23.0 (S—CH₂), 23.7 (CH₂—COOH), 34.7 (CH₂—Ar), 36.9 (CH₂—NH₂), 117.3 (C-2, C-5), 122.2 (C-1), 125.4 (C-6), 136.8 (C-3), 143.2 (C-4), 170.3 (acid); ES-MS: m/z 255.2 [M-H]⁻, 279.2 [M+Na−2H]⁻, 211.9 [M−catechol chain−H]⁺.

### Example 12

Catecholamine-Thioether Synthesis by Electrolysis

Dopamine 5-Mercaptopropanoic Acid (34)

Dopamine (33) (30 mg, 0.158 mmol) was dissolved in 80 ml of 0.1M HCl. The solution had a voltage of 2V applied across it between two pressed graphite bar electrodes and was vigorously stirred to prevent air bubble formation. The electrolysis was conducted over 2.5-3 hours and the initially colourless solution soon turned bright yellow and then bright orange. The formation of the coloured o-quinone was monitored by HPLC. Once maximum o-quinone formation had occurred, the solution then had 10% v/v 3-mercaptopropionic acid (412.6 μl, 0.473 mmol) added rapidly with vigorous stirring. The reaction was monitored and was left overnight as a precaution to ensure maximum product (34) formation. Yield: 14 mg (0.0545 mmol, 34%). Mp: decomposes. ¹H NMR: δ D₂O: 2.49 (2H, t, J=7.9 Hz, CH₂—S), 2.72 (2H, t, J=9.2 Hz, CH₂—N), 2.95 (2H, t, J=7 Hz, CH₂—Ar), 3.07 (2H, t, J=9.2 Hz, CH₂—COOH), 6.68 (1H, s, 6-H), 6.77 (1H, s, 2-H). ¹³C NMR: δ D₂O 28.6 (CH₂—S), 32.0 (CH₂—COOH), 34.1 (CH₂—Ar), 40.6 (CH₂—NH₂), 116.5 (2-C), 120.5 (5-C), 125.3 (6-C), 129.3 (1-C), 144 (3-C or 4-C), 144.5 (3-C or 4-C). ES-MS: 1:1 AcCN:H₂O 5V 258.9 [M+H]⁺.

Dopamine 5-Mercaptoundecanoic Acid (35)

Dopamine (33) (30 mg, 0.158 mmol) was dissolved in 0.2M HCl total 50% v/v acetonitrile and electrolysed at 2V with vigorous stirring for 2.5 hrs. The ortho-quinone formation was followed by HPLC and the current was observed to drop from 20 mA to 9 mA within 30 min period. 11-mercaptoundecanoic acid (103.7 mg, 0.475 mmol, in 6 ml of 50% v/v acetonitrile 0.2 M HCl total) was added rapidly to the vigorously stirring solution. Colour was observed to fade gradually until by 30 min. there is no significant colour left. Yield: 9.2 mg (0.025 mmol) 16%. ¹H NMR δ 1.21 (10H, main chain CH₂ of UDA), 1.36 (2H, UDA), 1.56 (4H, UDA), 2.36 (2H, CH₂—S), 2.87 (2H, CH₂—N), 2.88 (2H, CH₂—Ar), 3.22 (2H, CH₂—COOH), 6.78 (1H, Ar 5 or 6-H), 6.88 (1H, Ar 2-H). ¹³C NMR: δ 24.3, 28.3 (CH₂S), 32.1 (CH₂—COOH), 33.3, 33.9 (CH₂—Ar), 40.6 (CH₂—NH₂), 115.5 (2-C), 122.6 (5-C), 123.8 (6-C), 129.3(1-C), 143.0 (3-C or 4-C), 144.4 (3-C or 4-C), 179.1 (acid). ES-MS: (CH₃CN/H₂O) (370.6 M+H)⁺.

Nor-Epinephrine Mercaptopropanoic Acid (37)

Nor-epinephrine bitartrate (36) (40 mg, 0.125 mmol) was dissolved in 80 ml of 0.1M HCl and electrolysed at 2V until maximum conversion to ortho-quinone was observed (usually two hours). 3-Mercaptopropionic acid (327.5 μl of 1/10 solution in 0.1 M HCl, 0.375 mmol) was added with rapid stirring and the bright orange colour left the solution immediately. The reaction was stirred vigorously overnight. Yield: (14.0 mg, 0.0512 mmol, 41%) ¹H NMR: δ (D₂O) 2.67 (2H, t, J=7.2 Hz, S—CH₂), 3.15 (2H, m, CH₂—N), 3.27 (2H, m, CH₂—COOH), 4.55 (1H, s, CH—OH), 6.91 (1H, s, 5-H or 6-H), 7.07 (1H, s, 2-H). ¹³C NMR: δ (D₂O) 40.5 (CH₂—NH₂), 41.0 (CH—OH), 123 (6-C), 129 (1-C), 139 (3-C or 4-C). ES-MS: (20V, AcCN/H₂O) 274.3 [M+H]⁺.

Epinephrine Mercaptopropanoic Acid (39)

Epinephrine (38) (30 mg, 0.164 mmol) was dissolved in 0.1M HCl (80 ml) and electrolysed at 2V until maximum ortho-quinone formation was observed by HPLC. The solution then had 3-mercaptopropionic acid (428 μl of 1/10 solution in 0.1M HCl, 0.491 mmol) added rapidly to the rapidly stirring solution. The solution went from bright orange through green to a very deep green, almost black after 30 min. At 30 min. reaction the columning process was begun. Yield (%) 10.1 mg, 0.035 mmol (21%), Mp: decomposes. ¹H NMR δ: 1.31 (1H, m), 1.37 (1H, m), 2.75 (3H, s, NH—CH₃), 2.86 (2H, t, J=6.7 Hz, S—CH₂), 3.01 (2H, t, J=7.1 Hz, CH₂—COOH), 6.91-7.09 (2H, cluster, aromatics). ¹³C NMR: (δ) 16.7 (CH₂—S), 28.7 (CH₂—COOH), 42 (CH₂—Ar), 57.4 (CH₂—N), 108 (aromatic), 167 (aromatic). ES-MS: (CH₃CN:H₂O 1:1, −30V) 288.5 [M+H]⁺. (H₂O, 5V):214.5 [M-amine side chain+H]⁺, 306.3 [M+H₂O+H]⁺.

Antibody-Binding Studies

### Example 13

Biotination of Monoclonal Anti-Progesterone Antibody (Reaction Scheme 3)

Biotinyl-N-ε-aminocaproyl-N-hydroxysuccinimide ester (BcapNHS) was dissolved in dry DMF (5 mg/ml), and the monoclonal anti-progesterone antibody (100 μl) was dissolved into 0.1 M NaHCO₃ (1 ml). Add the BcapNHS solution in DMF (50 μl) to the above antibody solution in NaHCO₃ (1 ml); the solution was allowed to stand at room temperature for 2 hours without stirring.

The solution was then dialyzed overnight against 0.15 M NaCl (1 L) with several changes (>4 times); the last dialysis is performed against PBS/T (1 L) for at least 4 hours. Finally, the biotinylated antibody was further purified by passing through a PD-10 column to give 3.5 ml of pure antibody solution, which is stored at −20° C. for future uses.

### Example 14

Direct Antibody-Binding Performance on the Biosensor Surface (Reaction Scheme 1)

Immobilisations

Immobilization of progesterone-linker (11˜25 atoms linker)-OVA conjugates onto biosensor surfaces (activated CM-5 sensor chip) was done manually aiming for a minimum immobilisation of 2000RU. Progesterone-linker (11-atoms)-OVA conjugate was immobilised at pH 3.5 and progesterone-linker (25-atoms)-OVA conjugate at pH 4.0. Flow rates were 5 μL min⁻¹ and 2000 RU or greater was achieved in both cases. Final immobilisations were 2524 or 2208 RU for the above two conjugates respectively. The chip had a solution of OVA (5 μgmL⁻¹ in running buffer) passed over the surface to help to stabilise it (10 min. at 25 μLmin⁻¹). Immobilisation buffers were 10 mM sodium formate as previously (Steroids, 67, 2002, 565-572).

Binding Performance with Unmodified Antibody

Monoclonal anti-progesterone (unmodified) was passed over the surface to assess its binding (100 μgmL⁻¹ in running buffer, 3 min. injection at 20 μLmin⁻¹). This resulted in a binding of 654 RU for conjugate with 11-atoms linker, and 447 RU for the conjugate with 25-atoms linker. Regeneration was effected with 50 mM glycine buffer pH=1.5 (two pulses of 75 μL at 50 μLmin⁻¹ flow rate) and this were adequate for complete baseline return.

Binding Performance with Biotinated Antibody

Biotinylated monoclonal antibody was then passed over the surface (100 μgmL⁻¹ in running buffer, 3 min. injection at 20 μLmin⁻¹) and gave a binding of 406 or 142 RU for two conjugates respectively. This result indicates that the presence of biotin-linker units on the antibody has a significant effect on the degree of binding causing a 35% reduction for the conjugate having a 11-atoms linker, and a 60% reduction for the conjugate having a 25-atoms linker.

Binding Performance with Antibody-Nanogold Particle Conjugate

Biotinylated monoclonal antibody (100 μgmL⁻¹ in running buffer, 100 μL) was mixed 1:1 with 10 nm colloidal gold-streptavidin conjugate (Sigma S9059) and vortexed, and then incubated at room temperature for 10 min before injection (120 mL, 20 μLmin⁻¹). The resulting binding was 667 RU for the conjugate having an 11-atoms linker and 257 RU for the conjugate having a 25-atoms linker. This represents a signal enhancement of 64% or 82% for both conjugates respectively. Regeneration was again done using 50 mM glycine pH 1.5 as before and found to give complete return to baseline.

In order to determine the best antibody/gold volume ratio to use for competitive assay development, various ratios were optimised according to their antibody binding responses. The biotinylated monoclonal anti-progesterone was set at a concentration of 100 μgmL⁻¹. The ratios tested were 1:1 (80 μL mAb:80 μL gold), 1.67:1 (100 μL:60 μL), 3:1 (120 μL:40 μL), 7:1 (140 μL:20 μL) and 15:1 (150 μL:10 μL). The same testing was then done but with running buffer instead of gold colloid to determine the degree of gold signal enhancement at each ratio. The results are summarised below in Table 1 for the conjugate having an 11-atoms linker, and Table 2 for the conjugate with a 25-atoms linker.

TABLE 1

| | Volume Ratio mAb:gold | | | | |
|---|---|---|---|---|---|
| | 1 | 1.67 | 3 | 7 | 15 |
| mAb Only | 497.9 | | 802.3 | 731.9 | |
| mAb Gold | 796.3 | 890.5 | 929 | 957.1 | 893.5 |
| Enhancement | 298.4 | | 126.7 | 225.2 | |
| % Enhancement | 60 | | 16 | 31 | |

TABLE 2

| | Volume Ratio mAb:gold | | | | |
|---|---|---|---|---|---|
| | 1 | 1.67 | 3 | 7 | 15 |
| mAb Only | 184.4 | | 292.9 | 266.8 | |
| mAb Gold | 329.6 | 352.6 | 371.6 | 370.2 | 330.2 |
| Enhancement | 145.2 | | 78.7 | 103.4 | |
| % Enhancement | 79 | | 27 | 39 | |

The results clearly show that as the monoclonal antibody volume is increased without gold labelling, one observes an increase in response up until a ratio of 3:1 antibody:buffer after which it begins to decrease slowly. This pattern is seen for both conjugates the difference being the conjugate with a 25-atoms linker has much lower overall response than the other conjugate (11-atoms linker).

When considering the monoclonal antibody:gold colloid ratio, signal continues to increase up to a ratio of 7:1 mAb:gold though flattens out at the end and from 7:1 to 15:1 a slight decrease in response is observed for both conjugates. Once again the 4-3 response is much lower than that for 4-1.

The degree of gold colloid signal enhancement (expressed in absolute terms or as a percentage) is seen to peak at around 1.5:1 mAb:gold ratio and drop again until 3:1 after which a modest increase is observed up to 7:1. This suggests that gold enhancement is maximal at around 1.5:1 ratio and is less significant at higher antibody:gold ratios. Based on the signals obtained from the ratios above, the ratio giving largest overall signal considering both conjugates was selected as the ratio to use in development of a progesterone assay curve. The ratio selected was 7:1 mAb:gold.

### Example 15

Competitive Progesterone Immunoassay Using Progesterone-OVA Conjugate Surface and Antibody-Nanogold Conjugate as Flow Immunoreactant

A series of standard progesterone solutions were prepared in HBS buffer, at concentrations ranging from 0 to 1 μg/ml. Each sample (100 μl) was incubated with an equal volume (100 μl) of mixture of mAb (100 μgmL⁻¹):streptavidin/nanogold (10 nm) (7:1), incubating for 5 min at 25° C., and the resulting mixture (120 μl) passed over the chip surfaces for 6 minutes at a flow rate of 10 μlmin⁻¹. The regeneration of sensor surfaces was performed by two glycine buffer (50 mM, pH 1.5, 50 μlmin⁻¹, 2 min) pulses. The same procedure was carried out three times for each concentration.

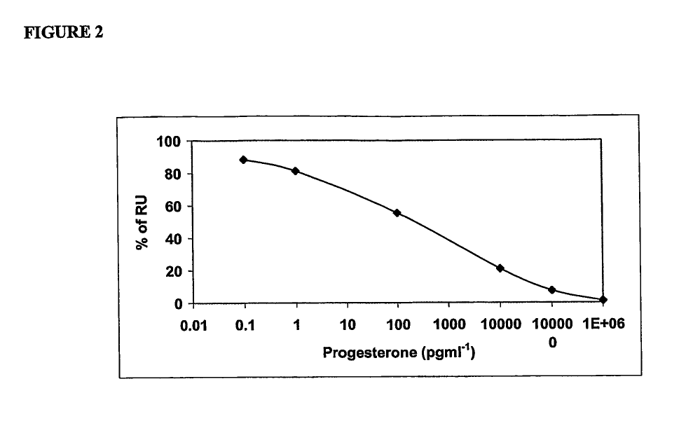

A plot of concentrations of free progesterone versus percentage (%) bound of RU relative to zero progesterone concentration provides two standard curves for two progesterone-OVA conjugates. The standard curve for progesterone-OVA conjugate with a 25-atoms linker is shown in FIG. 2. The assays for both conjugates demonstrate a very broad detection region from 1 μgmL⁻¹ to <0.1 pgmL⁻¹. The lowest detection limit is assessed as <0.1 pgmL⁻¹ by both the 90% bound and zero-three standard deviations method, and the 50% bound values are both given in Table 3

TABLE 3

| | 50% Bound | Detection Limit |
|---|---|---|
| Conjugate | (pgmL-1) | (pgmL-1) |
| 11-atoms linker | 1300 | 0.1 |
| 25 atoms linker | 89 | 0.1 |

### Example 16

Biotination of Monoclonal Anti-Progesterone Antibody (Reaction Scheme 3)

Biotinamidocaproate-N-hydroxysuccinimide ester (BcapNHS) (Sigma Aldrich B-2643) was dissolved in dry DMF to make a 5 mg/mL solution. Monoclonal anti-progesterone (100 μL) was added to 0.1 M sodium bicarbonate solution (900 μL) and the BcapNHS solution was added (25 μL in 1 mL of 0.1 M sodium bicarbonate) drop-wise to the stirring antibody solution. The solution was stirred for 5 min. before leaving without stirring at room temperature for two hours. The solution was then dialyzed against 0.15 M NaCl at 4° C. for four changes (one overnight) and then four changes of PBS/T (one overnight). The solution was then passed through a PD-10 column and protein concentration determined by assumption of negligible loss of antibody, as the BCA method of protein concentration determination was found to be unreliable due to the effects of modifying the antibody with biotin and thus changing the numbers of free lysine residues. Antibody was stored frozen until use. SPR binding studies showed ≧85% binding integrity relative to unmodified antibody.

### Example 17

Preparation of Anti-IgG-Gold Conjugates

Gold colloids of 25 nm, 55 nm and 70 nm were prepared by the method of citrate reduction (Nature 1973, 241, 20-23) with some modifications to the citrate loadings. All sols were produced at a 0.01% w/v HAuCl₄ loading. The colloid sizes were determined by photon correlation spectroscopy (PCS) using a Malvern Zetasizer. The Z_{avg} parameter was used for the 25 nm of colloid and the intensity parameter for the others. 30 replicates were done for the 25 nm colloid and six and five determinations each with 10 sub-runs was done for the other two respectively. The Zetasizer determinations were validated by measuring a 20 nm commercial gold sol (Sigma G1652) which gave 23.0±1.0 nm, n=30 compared to 19±2.1 nm by TEM. Five-fold concentrated gold sols were prepared by adding PEG-400 3% v/v to the sol and centrifuging at 14 k×g for 30 min before removing supernatant and reconstituting in deionized water with sonication.

Anti-IgG-gold conjugates were produced by altering the pH of the sol to 8.5 with dilute NaOH and adding anti-rat IgG at 8 mg/mL in deionized water (pH=8.5), at 10% v/v to the colloid with vortex agitation. The colloid was shaken for 5 min., stored at 4° C. overnight and then blocked with 20% w/v BSA, 1% v/v as for the antibody.

### Example 18

Surface Immobilisation (Reaction Scheme 2)

A stock solution in DMF of 100 mg/mL of compound 6 was prepared. The stock was diluted 1/100 in PBS/T pH=9.0 for injection. A new BIAcore CM5 chip (BIAcore, Uppsala, Sweden) had flow cell two activated with N-ethyl-N-(3-dimethylaminopropyl)-carbodiimide (EDC) and NHS (150 μL of each transferred to a vial and then 200 μL mixed and 50 μL injected at 5 μL/min). The progesterone-PEG-amine solution was then quick injected at 5 μL/min, 100 μL. The surface was then deactivated with ethanolamine (50 μL, 5 μL/min) to give an immobilization binding of 638.9 RU. Flow cell one was activated and deactivated as a blank flow cell analogously to flow cell two. Flow cell three was immobilized to give a 1333.8RU response. The surfaces were then washed with three pulses of 50 mM NaOH at 15 μL at 5 μL/min.

The immobilized surface of one chip has shown a very stable surface as demonstrated by more than 1100 binding and regeneration cycles without any appreciable drop in antibody binding capacity and significant baseline shifts.

### Example 19

Biotin/Streptavidin Mediated Inhibition immunoassays

Biotinylated monoclonal antibody (100 μg/mL) was mixed with 10 nm-gold-streptavidin conjugate in volume ratios of 0.5, 1, 5, 3, 7 of antibody/gold and incubated at room temperature for 2 h. The mixture was then injected over the surface in a 1:1 dilution with running buffer (60 μL, 20 μl/min) and the surface regenerated with two pulses of 10% v/v acetonitrile in 50 mM NaOH, five replicates done in a BIAcore wizard program. The assay was constructed in the same way but using progesterone standards of 0, 10 fg/mL, 1, 10, 100 pg/mL, 1, 10, 100 ng/mL and 1 μg/mL instead of buffer. Antibody and standard were incubated at room temperature for 5 min before injection. The 20 nm-gold-streptavidin colloid was used to construct an assay as for the 10 nm colloid but using 0.2 M ethylene glycol in the 7:1 antibody/gold preparation and using progesterone standards of 0, 10, 100 fg/mL, 1, 10, 100, 500 pg/mL, 1, 10, 100 ng/mL.

Gold dilution binding tests were done for a sequential injection assay by quick injecting biotinylated antibody (50 μg/mL, 60 μL, 20 μL/min) followed immediately by a quick injection of 10 nm-gold-streptavidin (30 μL, 20 μL/min). After a 180 s delay the surface was regenerated with three pulses of 20% v/v acetonitrile 200 mM NaOH (20 μl, 20 μl/min.). This was done for five replicates of 0.25, 0.15, 0.10, 0.05, 0.02, 0.01 dilution of gold in 0.2 M ethylene glycol total concentration and 10% w/v BSA total concentration. Antibody binding curves were established by setting the flow rate to 20 μl/min. and quick injecting biotinylated antibody (60 μL) followed immediately by 10 nm-gold-streptavidin (0.15 dilution, 1% v/v PEG-400), a 180 s wait and then regeneration (three×20% v/v acetonitrile, 200 mM NaOH) using antibody concentrations of 0, 5, 10, 15, 25, 35, 50 μg/mL with five replicates each. Assays were determined by mixing 70 μL of biotinylated monoclonal antibody (concentrations of 5-30 μg/mL) with 70 μL of progesterone (0, 100 fg/mL, 1 or 5, 10, 20, 50, 100, 500 pg/mL, 1, 10, 100 ng/mL) and incubating at 25° C. for 5 min before injection (60 μL, 20 μL/min throughout) immediately followed by a quick inject of 10 nm-gold-streptavidin (30 μL, with either 10% w/v BSA, 0.2 M ethylene glycol total concentrations or 1% v/v PEG-400) followed by regeneration as for the antibody binding.

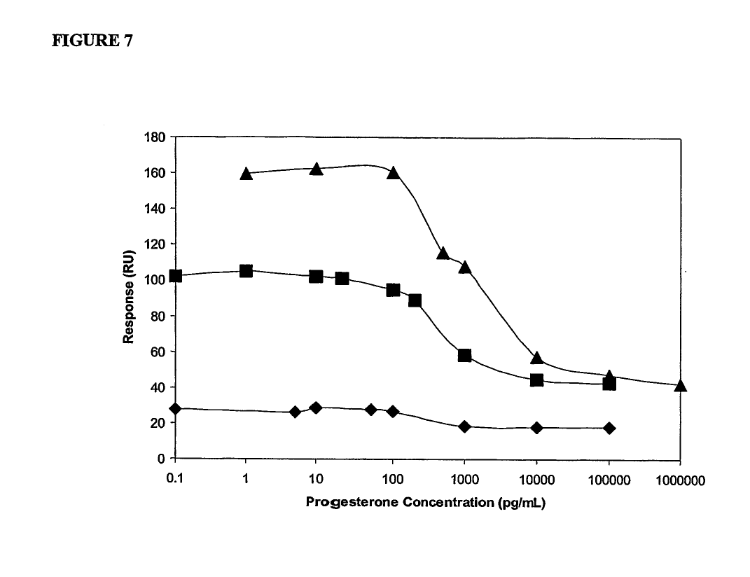

Assays constructed around this format showed a LOD that was dependent upon the concentration of monoclonal antibody used. The LOD were 150±49, 23.1±4.4 and 104±40 pg/mL (Table 4) for concentrations of 15, 7.5 and 2.5 μg/mL of biotinylated antibody respectively (FIG. 7).

TABLE 4

| | | | | Sensi- | |
|---|---|---|---|---|---|
| | | | | tivity | Enhance- |
| Assay | mAb | LOD | IC-50 | (RU | ment |
| Format | (μg/mL) | (pg/mL) | (pg/mL) | mL/ng) | Ratio |
| mAB only | 43.75 | 449 | 1514 | 49 | n/a |
| Pre-incubation | 43.75 | 143 ± 35 | 1670 ± 100 | 57 | 1 |
| (10 nm) | | | | | |
| Pre-incubation | 43.75 | 198 ± 57 | 1910 ± 150 | 28 | 1 |
| (20 nm) | | | | | |
| Sequential gold | 15 | 150 ± 49 | 1000 ± 145 | 32 | 2 |
| (10 nm) | | | | | |
| Sequential gold | 7.5 | 23.1 ± 4.4 | 460 ± 16 | 40 | 2 |
| (10 nm) | | | | | |
| Sequential gold | 2.5 | 104 ± 40 | 314 ± 21 | 12 | 2 |
| (10 nm) | | | | | |
| Anti-IgG | 3 | 20.1 ± 4.0 | 242.8 ± 5.1 | 99 | 8 |
| Anti-IgG | 25 | 246 ± 4.1 | 810 ± 72 | 226 | 8 |
| Anti-IgG/gold | 1.5 | 8.6 ± 3.9 | 151.7 ± 2.1 | 308 | 13 |
| (25 nm) | | | | | |

### Example 20

Anti-IgG Mediated Inhibition Immunoassays

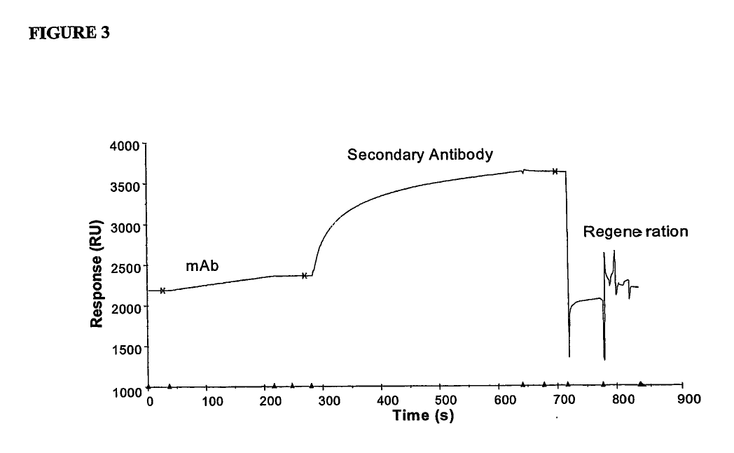

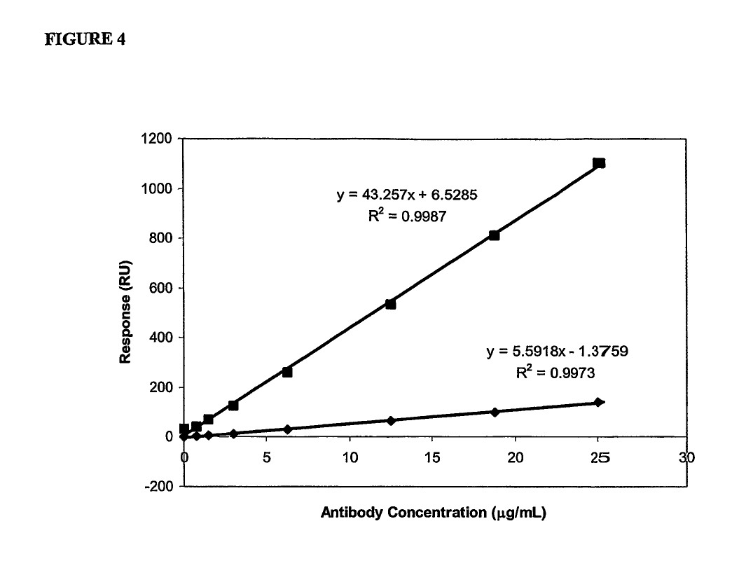

Anti-IgG enhancement curves were prepared by quick injecting monoclonal antibody (25 μg/mL, 60 μL, 20 μL/min) immediately followed by anti-rat IgG (60 μL, 10 μL/min) and then regeneration (one pulse as above) (FIG. 3). Anti-IgG concentrations of 0, 50, 100, 200, 400, 600, 800 μg/mL were used, five replicates of each. Antibody binding curves were prepared as for the enhancement curves but keeping secondary antibody concentration fixed at 800 μg/mL and varying concentration of monoclonal antibody: 0, 0.75, 1.5, 3, 6.25, 12.5, 18.75, 25 μg/mL. Assays were set up by the same method as for the biotin/streptavidin sequential assays but using anti-rat IgG (800 μg/mL) in place of the gold and a 30s wait before regeneration with one pulse of regeneration solution. Progesterone standards of 0, 0.1, 1, 5, 10, 50, 100 pg/mL, 1, 10, 50 ng/mL were run with five replicates. In this experiment we found that if anti-IgG is used at a high concentration (800 mg/mL) then one observes signal enhancements of 8.1-fold (FIG. 4).

Antibody binding plots were prepared as above but using anti-IgG-gold 25 nm (0.5 dilution in deionized water, 10% v/v PEG-400, conjugate produced using 200 μg/mL IgG 1 mL to 10 mL of colloid, pH=8.1, three-fold concentrated by centrifugation at 4° C. after blocking with BSA (10% w/v, 3.66 mL per 10 mL colloid), unbound IgG removed in the centrifugation). There is a 180 s wait after gold and then regeneration with one pulse.

Bindings of 25, 45, 55, and 70 nm colloids synthesized as mentioned above and used as is or five times concentrated, were determined by injection of monoclonal antibody (25 μg/mL, 60 μL, 20 μL/min) followed by IgG-gold (undiluted, 60 μL, 10 μL/min) and regenerated as before. Each binding was determined in triplicate. Antibody binding plots were determined as before for the 25 nm gold-secondary antibody, 5× concentrated, using monoclonal antibody concentrations of 0, 1, 2, 5, 10, 15, 25 μg/mL and with the gold having a 1% v/v PEG-400 loading. Assay curves for the 25 nm-gold-IgG were prepared as before using progesterone concentrations of 0, 1, 10, 50, 100 pg/mL, 1, 10 ng/mL.

When the assay applied at low monoclonal antibody concentration (1.5 μg/mL), the assay showed 13-fold enhancement (and a LOD of 8.6±3.9 pg/mL. The sensitivity of the assay has increased to three-fold from that of the anti-IgG only format at 3 μg/mL and the whole assay curve has clearly shifted to lower concentration as seen in both the LOD and IC₅₀ values.

### Example 21

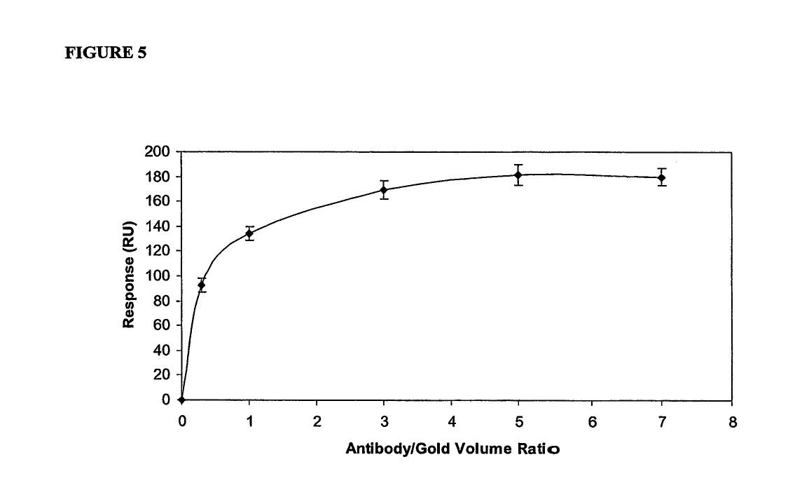

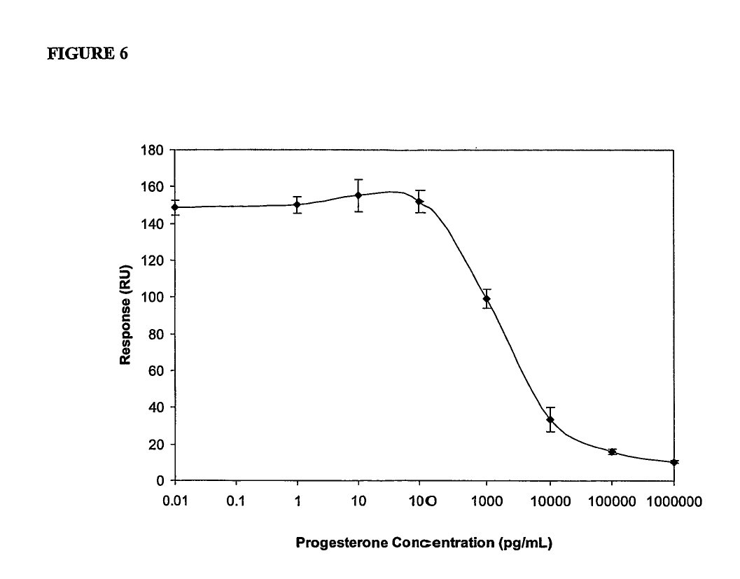

Biotin/Streptavidin Mediated Assays (FIGS. 5 and 6).

Biotinylated monoclonal antibody (100 μg/mL) was mixed with 10 nm-gold-streptavidin conjugate in volume ratios of 0.5, 1, 5, 3, 7 of antibody/gold and incubated at room temperature for 2 h. The mixture was then injected over the surface in a 1:1 dilution with running buffer (60 μL, 20 μl/min) and the surface regenerated with two pulses of 10% v/v acetonitrile in 50 mM NaOH, five replicates done in a BIAcore wizard program (FIG. 5). The assay was constructed in the same way but using progesterone standards of 0, 10 fg/mL, 1, 10, 100 pg/mL, 1, 10, 100 ng/mL and 1 μg/mL instead of buffer (FIG. 6). Antibody and standard were incubated at room temperature for 5 min before injection.

The above examples are illustrations of practice of the invention. It will be appreciated by those skilled in the art that the invention can be carried out with numerous modifications and variations. For example the haptens, the linkers, the antibodies and the concentrations used may all be varied.

1. A method for detecting a hapten in a sample comprising the steps of:
a) providing a sample potentially containing the hapten;
b) providing a pre-determined amount of a first moiety, said first moiety being bound to a signaller and separated therefrom by a first linker, which first moiety is either:
i) a binding partner that specifically binds to the hapten of interest, or
ii) the hapten of interest or an analogue thereof;
wherein said signaller is a macromolecule or a nanoparticle providing high mass signal;
c) providing a flow of a) and b) separately or together to an immobilised second moiety, said second moiety being bound to the surface of a sensor and separated therefrom by a second linker, which second moiety is either:
i) a binding partner that specifically binds to the hapten of interest, or
ii) is the hapten of interest or an analogue thereof,
providing that when the first moiety is a binding partner, the second moiety is a hapten or hapten analogue and when the first moiety is a hapten or hapten analogue, the second moiety is a binding partner; and
c) detecting the amount of first moiety bound to second moiety.

2. The method as claimed in claim 1 for detecting a hapten in a sample comprising the steps of:
a) providing a sample potentially containing a hapten of interest;
b) providing a pre-determined amount of a binding partner that specifically binds to the hapten of interest, said binding partner being bound to a signaller and separated therefrom by a first linker wherein said signaller is a large protein or a nanoparticle providing a high mass signal;
c) providing a flow of separately or together of a) and b) to an immobilised hapten of interest or an analogue thereof, said hapten or analogue thereof being bound to the surface of a sensor and separated therefrom by a second linker; and
d) detecting the amount of binding partner bound to said immobilised hapten or an analogue thereof.

3. The method as claimed in claim 1 for detecting a hapten in a sample comprising the steps of:
a) providing a sample potentially containing a hapten of interest;
b) providing a predetermined amount of the hapten of interest or an analogue thereof, said hapten or analogue thereof being bound to a signaller and separated therefrom by a first linker wherein said signaller is a large protein or a nanoparticle providing a high mass signal;
c) providing a flow of the resultant mixture of a) and b) to an immobilised binding partner that specifically binds to the hapten of interest, said binding partner being bound to the surface of a sensor and separated therefrom by a second linker; and
d) detecting the amount of hapten or analogue thereof bound to said immobilised binding partner.

4. A method for detecting a hapten in a sample using a rapid flow-through inhibition assay format comprising the steps of:
a) Providing an immobilised hapten derivative on the surface of an optical biosensor chip, the hapten molecule being separated from the surface by a first linker;
b) Mixing high molecular weight detecting molecules with sample analytes to form immuno-complexes, and then flow-through of the mixing solution containing excess free antibodies to bind to the sensor surface;
c) Further binding enhancement performed by flowing-through onto the sensor surface with a solution containing a specially designed bio-conjugate, in which by employing a suitable linker (second linker), a moiety to specifically recognise a detecting molecule such as an antibody is linked at one end of the conjugate, and the other end of the conjugate is attached to a large protein or/and a nano-particle for high mass signal enhancement;
d) Detecting the amount of binding partner bound to the hapten derivative thereof.

5. The rapid flow-through competition method of claim 1 for detecting a hapten in a sample comprising the steps of:
a) Providing immobilised detecting molecules onto the biosensor surface with a linker (first linker) between a bio-material as an attachment intermediate and the detecting molecule;
b) Mixing sample analytes with a hapten conjugate, in which a protein or/and a nano-particle is linked to the hapten molecule with a linker (second linker) and having a nano-distance (nm) between the protein/nano-particle and the hapten molecule to reduce steric hindrance;
c) Flowing through the mixture of hapten conjugate and sample analyte solution onto the sensor surface for binding competition to limited detecting molecules such as antibodies on the surface of the sensor;

6. The method as claimed in claim 1 wherein the hapten is selected from the group comprising carbohydrates, polynucleotides, steroids, steroid analogues, polypeptides, drugs, neurotransmitters, hormones and toxins.

7. The method as claimed in claim 6 wherein the hapten is a steroid.

8. The method as claimed in claim 7 wherein the steroid is progesterone.

9. The method as claimed in claim 1 wherein the binding partner is selected from antibody molecules and fragments of antibody molecules retaining hapten-binding ability.

10. The method as claimed in claim 1 wherein the surface is a surface of an optical biosensor chip.

11. The method as claimed in claim 1 wherein the hapten is a steroid and binding of the hapten to the linker occurs at the 4-position of the A-ring structure.

12. The method as claimed in claim 1 wherein the hapten is progesterone.

13. The method as claimed in claim 1 wherein the first linker and second linker are each independently 10 to 50 atoms in length.

14. The method as claimed in claim 1 wherein the first linker and the second linker are independently selected from (a) a carbon-based chain; (b) a carbon-chain containing one or more heteroatoms; (c) a carbon-chain with substituted groups; (d) an amino acid chain, amino acid fragments incorporated into the chain, or multiple amino-acid fragments chain by homologation; (e) an oligoethylene glycol or a polyethylene glycol chain; (f) a chain having one or more sites of unsaturation such as alkenyl; and (g) a nucleic acid chain; or (h) a polysaccharide chain.

15. The method as claimed in claim 1 wherein the hapten is a steroid and the linker between steroid and the surface is an oligoethylene glycol or a polyethylene glycol chain.

16. The method as claimed in claim 1 wherein the signaller is a nanoparticle.

17. The method as claimed in claim 1 wherein the signaller is an immunogold particle.

18. The Surface Plasmon Resonance based immunoassay format method comprising the steps:
(a) chemically immobilising a hapten or hapten conjugate onto the optical biosensor surface through a linker molecule (the second linker) with or without using a hapten attachment intermediate,
(b) mixing a fixed concentration of a binding partner—(the first linker)—nanoparticle conjugate in buffer with each of a series of standard free solution or a sample hapten solution and incubating for a few minutes,
(c) injecting the above mixture or the remaining binding partner in equilibrium solution onto the hapten—biosensor surfaces, and measuring binding partner responses,
(d) injecting regeneration buffer onto the biosensor surface to remove binding partner—(the first linker)—nanoparticle conjugate,
(e) plotting concentrations of free hapten versus average response (resonance units) of binding partner—(the first linker)—nanoparticle conjugate to provide an assay standard curve from which determining the concentration of unknown sample hapten when using the same method.

19. The method as claimed in claim 4 wherein the hapten is selected from the group comprising carbohydrates, polynucleotides, steroids, steroid analogues, polypeptides, drugs, neurotransmitters, hormones and toxins.

20. The method as claimed in claim 19 wherein the hapten is a steroid.

21. The method as claimed in claim 20 wherein the steroid is progesterone.

22. The method as claimed in claim 19 wherein the binding partner is selected from antibody molecules and fragments of antibody molecules retaining hapten-binding ability.

23. The method as claimed in claim 19 wherein the hapten is a steroid and binding of the hapten to the linker occurs at the 4-position of the A-ring structure.

24. The method as claimed in claim 19 wherein the hapten is progesterone.

25. The method as claimed in claim 19 wherein the first linker and second linker are each independently 10 to 50 atoms in length.

26. The method as claimed in claim 19 wherein the first linker and the second linker are independently selected from (a) a carbon-based chain; (b) a carbon-chain containing one or more heteroatoms; (c) a carbon-chain with substituted groups; (d) an amino acid chain, amino acid fragments incorporated into the chain, or multiple amino-acid fragments chain by homologation; (e) an oligoethylene glycol or a polyethylene glycol chain; (f) a chain having one or more sites of unsaturation such as alkenyl; and (g) a nucleic acid chain; or (h) a polysaccharide chain.

27. The method as claimed in claim 4 wherein the hapten is a steroid and the linker between steroid and the surface is an oligoethylene glycol or a polyethylene glycol chain.

28. The method as claimed in claim 4 wherein the signaller is a nanoparticle.

29. The method as claimed in claim 4 wherein the signaller is an immunogold particle.

30. The method as claimed in claim 4 wherein detecting molecules are removed by rapid on-line flow-through regeneration to allow multiple measurements.

31. The method as claimed in claim 4 wherein a standard curve is prepared from solutions with a series of known analyte concentrations, and the concentrations of analyte in unknown samples are then derived from the standard curve.
